(19) United States
(12) Patent Application Publication
(10) Pub. No.: US 2014/0056898 A1
(43) Pub. Date: Feb. 27, 2014

(54) COMBINATION THERAPIES COMPRISING ANTI-ERBB3 AGENTS

(71) Applicants: Bo Zhang, Lynnfield, MA (US); Charlotte McDonagh, Winchester, MA (US); Alexandra Huhalov, Cambridge, MA (US)

(72) Inventors: Bo Zhang, Lynnfield, MA (US); Charlotte McDonagh, Winchester, MA (US); Alexandra Huhalov, Cambridge, MA (US)

(21) Appl. No.: 14/001,448

(22) PCT Filed: Feb. 24, 2012
(86) PCT No.: PCT/US12/26602

§ 371 Date: Oct. 23, 2013

Related U.S. Application Data
(60) Provisional application No. 61/446,326, filed on Feb. 24, 2011.
(60) Provisional application No. 61/470,848, filed on Apr. 1, 2011.

(51) Int. Cl. A61K 39/395; A61K 31/138; A61K 31/4196; A61K 31/337; A61K 31/517; A61K 31/7068; A61K 33/24; A61K 31/565; A61K 45/06

(52) U.S. Cl. CPC A61K 39/3955; A61K 31/565; A61K 31/138; A61K 31/4196; A61K 45/06; A61K 31/517; A61K 31/7068; A61K 33/24; A61K 31/337
USPC 424/136.1; 435/375

(57) ABSTRACT

Disclosed are methods and compositions for inhibiting the growth of a tumor (e.g., a malignant tumor) in a subject. In particular, combination therapies for treating a tumor in a subject by co-administering either i) an effective amount of an anti-estrogen agent or ii) an effective amount of a receptor tyrosine kinase inhibitor and an effective amount of a bispecific anti-ErbB2/anti-ErbB3 antibody, and optionally an effective amount trastuzumab. Also disclosed is a bispecific anti-ErbB2/anti-ErbB3 antibody for use in the therapy of a tumor in combination with either i) an anti-estrogen agent or ii) a receptor tyrosine kinase inhibitor, and optionally in use with trastuzumab.

### FIELD OF THE INVENTION

The various aspects of the invention disclosed herein relate to methods and compositions for the treatment of cancers.

### BACKGROUND OF THE INVENTION

Approximately 75% of breast cancers are estrogen receptor (ER) positive. Other cancers are also ER positive (ER+). Estrogen receptors mediate intracellular signaling that can increase the frequency of cell division and drive tumor growth. Although anti-endocrine therapies such as tamoxifen, fulvestrant, and letrozole have demonstrated significant efficacy in treating ER+ breast cancer patients, intrinsic or acquired resistance to such therapies has limited their success.

The prevalence of amplification of the human epidermal growth factor receptor 2 (HER2, or ErbB2) in breast cancer and other cancers has resulted in the research and development of drugs that have ErbB2 as a therapeutic target. Although both the anti-ErbB2 monoclonal antibody trastuzumab and the ErbB1/ErbB2 dual receptor tyrosine kinase inhibitor lapatinib have met with success in the clinic, many patients fail to benefit from these drugs. Additionally, the majority of patients with tumors that initially respond will eventually recrudesce after extended treatment using these therapies.

The ErbB2/ErbB3 heterodimer is the most potent ErbB receptor pairing with respect to strength of interaction, impact on receptor tyrosine phosphorylation, and effects on downstream signaling through mitogen activated protein kinase and phosphoinositide-3 kinase pathways. Heregulin is the primary ligand for ErbB3, and activates signaling by ErbB2/ErbB3 heterodimers. Current ErbB2-targeted therapies do not effectively inhibit heregulin activated signaling. MM-111 is a bispecific anti-ErbB2/anti-ErbB3 antibody that abrogates heregulin binding to ErbB2/ErbB3 and inhibits heregulin activation of ErbB2/ErbB3 without significantly affecting ErbB2 biological activity. In preclinical models of HER-2+ gastric, breast, ovarian and lung cancers, MM-111 inhibits ErbB3 phosphorylation, cell cycle progression, and tumor growth.

Thus, a need exists for therapies and therapeutic strategies providing improved inhibition of ErbB3 activation (e.g., ligand-induced activation) as well as for therapies and therapeutic strategies providing improved inhibition of estrogen receptor signaling activity or of ErB1 and ErbB2 receptor signaling activity.

In the treatment of cancers, the co-administration of pluralities of anti-cancer drugs (combination therapy) often provides better treatment outcomes than monotherapy. Such outcomes can be subadditive, additive, or superadditive. That is to say that the combined effects of two anti-cancer drugs, each of which provides a quantifiable degree of benefit, can be less than, equal to, or greater than the sum of the benefits of each drug. For example, two drug, each of which when used alone to treat a lethal cancer provides an average one year extension of progression free survival, could together provide a <24 month extension (e.g., an 18 month extension), about a 24 month extension, or a >24 month extension (e.g., a 30 month extension) of progression free survival. Typically, combination therapies for cancer treatment provide significantly subadditive outcomes. Outcomes that are near additive, additive, or superadditive are most desirable, but only occur rarely. In addition, many drugs are known to alter the bioavailability, or otherwise affect the safety profile of other drugs when both drugs are co-administered. As new drugs are first used in combination therapies, unforeseen, hazardous drug-drug interactions may be observed that result in drug-drug interaction-mediated toxicity in the patient.

Thus approaches for safely administering combination therapies comprising administration of ErbB2/ErbB3 heterodimer-targeted agents for cancer treatment, and especially combinations that yield near-additive, additive, or superadditive outcomes are needed.

### SUMMARY OF THE INVENTION

Provided herein are methods and compositions effective for the inhibition of ErbB3 activation and also effective for the inhibition of estrogen receptor activation. Also provided are methods and compositions effective for the inhibition of ErbB3 activation and also effective for the inhibition of ErB1 and/or ErbB2 activation. These methods and compositions are useful for the treatment of tumors, e.g., malignant tumors, as well as for the treatment of other cancers.

In a first embodiment, a method of treating a subject with a malignant tumor is provided, where the tumor is an ErbB2 expressing or ErbB2 over-expressing tumor (e.g., HER⁺⁺ or HER⁺⁺⁺ tumors) and the tumor may be a melanoma, clear cell sarcoma, head and neck, endometrial, prostate, breast, ovarian, gastric, colon, colorectal, lung, bladder, pancreatic, salivary gland, liver, skin, brain or renal tumor. The method comprises co-administering to the subject either an effective amount of an anti-estrogen agent or an effective amount of a receptor tyrosine kinase inhibitor, in combination with an effective amount of an anti-ErbB3 agent, e.g., a bispecific anti-ErbB2/anti-ErbB3 antibody (e.g., the antibody comprising the amino acid sequence set forth in SEQ ID NO:1) and optionally an effective amount of trastuzumab.

In one aspect, the combination of the bispecific anti-ErbB2/anti-ErbB3 antibody and either the effective amount of an anti-estrogen agent or the effective amount of the receptor tyrosine kinase inhibitor, and optionally the effective amount of trastuzumab, is characterized as follows: when a first tissue culture medium is prepared comprising the bispecific anti-ErbB2/anti-ErbB3 antibody (e.g., the antibody comprising the amino acid sequence set forth in SEQ ID NO:1) at a first concentration and either the anti-estrogen agent at a second concentration or the receptor tyrosine kinase inhibitor (e.g., lapatinib) at a third concentration (wherein each concentration is the same or different as each other concentration), and the medium is contacted with cancer cells of a cell line in a cell culture, cell growth or cell proliferation or production of pErbB3 or production of pAKT in the cells is inhibited, or the percentage of cells in the culture that are apoptotic is increased. In certain aspects, cell growth or cell proliferation or production of pErbB3 or production of pAKT in the cells is inhibited, or the percentage of cells in the culture that are apoptotic is increased to a greater degree than cell growth, or cell proliferation or production of pErbB3 or production of pAKT in the cells is inhibited, or percentage of cells in the culture that are apoptotic is increased, to a lesser degree when cancer cells of the cell line in a cell culture are contacted with each of a second medium that is essentially the same as the first medium except that it does not comprise a bispecific anti-ErbB2/anti-ErbB3 antibody, and a third medium that is essentially the same as the first medium except that it does not comprise any anti-estrogen agent and it does not comprise any receptor tyrosine kinase inhibitor.

In another aspect, all effective amounts are either mouse effective amounts or human effective amounts. In another aspect, all effective amounts are mouse effective amounts and the combination of the bispecific anti-ErbB2/anti-ErbB3 antibody (optionally the antibody comprising the amino acid sequence set forth in SEQ ID NO:1) and either the effective amount of an anti-estrogen agent or the effective amount of the receptor tyrosine kinase inhibitor, is characterized as follows: when co-administered to BT474-M3 xenograft tumor bearing mice with a tumor of a measured volume, the combination is more effective at inhibiting tumor volume increase after 32 days of co-administration than is the mouse effective amount of the bispecific anti-ErbB2/anti-ErbB3 antibody administration without the co-administration of either the effective amount of an anti-estrogen agent or the effective amount of the receptor tyrosine kinase inhibitor. In another aspect, a mouse effective amount of trastuzumab is co-administered with the bispecific anti-ErbB2/anti-ErbB3 antibody.

In a second embodiment, a bispecific anti-ErbB2/anti-ErbB3 antibody (optionally the antibody comprising SEQ ID NO:1) is provided for use in combination therapy of a cancer (optionally a melanoma, clear cell sarcoma, head and neck, endometrial, prostate, breast, ovarian, gastric, colon, colorectal, lung, bladder, pancreatic, salivary gland, liver, skin, brain or renal tumor), where the combination therapy comprises concomitant use of either an anti-estrogen agent or a receptor tyrosine kinase inhibitor and optionally comprises concomitant use of trastuzumab.

In a third embodiment, an aqueous solution is provided comprising a bispecific anti-ErbB2/anti-ErbB3 antibody (optionally the antibody comprising the amino acid sequence set forth in SEQ ID NO:1) at a first concentration and either an anti-estrogen agent at a second concentration or a receptor tyrosine kinase inhibitor at a third concentration. In certain aspects, when a first tissue culture medium is prepared comprising the bispecific anti-ErbB2/anti-ErbB3 antibody at the first concentration and either the anti-estrogen agent at the second concentration or the receptor tyrosine kinase inhibitor at the third concentration, and the medium is contacted with cancer cells of a cell line in a cell culture, cell growth or cell proliferation or production of pErbB3 or production of pAKT in the cells is inhibited, or percentage of cells in the culture that are apoptotic is increased. In certain aspects, cell growth or cell proliferation or production of pErbB3 or production of pAKT in the cells is inhibited, or the percentage of cells in the culture that are apoptotic is increased, to a lesser degree when cells of the cell line in a cell culture are contacted with a second tissue culture medium that is essentially the same as the first medium of except that it does not comprise any anti-estrogen agent and it does not comprise any receptor tyrosine kinase inhibitor. In another aspect, cell growth or cell proliferation or production of pErbB3 or production of pAKT in the cells is inhibited, or the percentage of cells in the culture that are apoptotic is increased, to a lesser degree when cells of the cell line in a cell culture are contacted with a third tissue culture medium that is essentially the same as the first medium of except that it does not comprise any bispecific anti-ErbB2/anti-ErbB3 antibody.

In another aspect, the aqueous solution is blood plasma in a subject, and the subject does not experience a toxicity that is sufficiently harmful to require a change in a therapy being administered to the subject, which toxicity is mediated by a drug-drug interaction in the subject between the bispecific anti-ErbB2/anti-ErbB3 antibody and the anti-estrogen agent or the receptor tyrosine kinase inhibitor.

In another aspect, the aqueous solution further comprises trastuzumab at a fourth concentration, and the medium also comprises trastuzumab at the fourth concentration.

In another aspect, the method, combination therapy, or aqueous solution does not comprise an aromatase inhibitor or an estrogen receptor antagonist. In one embodiment the method, combination therapy, or aqueous solution comprises nab-paclitaxel.

In each embodiment and aspect thereof above, the anti-estrogen agent may be an estrogen receptor antagonist (e.g., fulvestrant or tamoxifen) or an aromatase inhibitor (e.g., wherein the aromatase inhibitor is letrozole, exemestane, anastrozole, aminoglutethimide, testolactone, vorozole, formestane, or fadrozole. Preferably the aromatase inhibitor is letrozole. Also in each embodiment and aspect thereof above, the receptor tyrosine kinase inhibitor is erlotinib, afatinib, dasatinib, gefitinib, imatinib, pazopinib, lapatinib, sunitinib, nilotinib or sorafenib. Preferably the receptor tyrosine kinase inhibitor is lapatinib. Also in each embodiment and aspect thereof above, the bispecific anti ErbB2/anti-ErbB3 antibody is the A5-HSA-ML3.9, ML3.9-HSA-A5, A5-HSA-B1D2, B1D2-HSA-A5, B12-HSA-B1D2, B1D2-HSA-B12, A5-HSA-F5B6H2, F5B6H2-HSA-A5, H3-HSA-F5B6H2, F5B6H2-HSA-H3, F4-HSA-F5B6H2, F5B6H2-HSA-F4, B1D2-HSA-H3, H3-HSA-B1D2, or the antibody comprising the amino acid sequence set forth in SEQ ID NO:1. Each embodiment and aspect thereof above may also further comprise use of capecitabine and/or cisplatin.

In each embodiment and aspect thereof above, one or more of a)-x) that follow may optionally apply: a) the cell line is BT474-M3; b) the culture is a spheroid culture, c) paclitaxel or another taxane or another chemotherapeutic drug is co-administered, optionally in accordance with the manufacturer's directions, d) the anti-estrogen agent is administered in accordance with the manufacturer's directions, e) the receptor tyrosine kinase inhibitor is administered in accordance with the manufacturer's directions, f) the trastuzumab is administered in accordance with the manufacturer's directions, g) the co-administration of the bispecific anti-ErbB2/anti-ErbB3 antibody with an anti-estrogen agent produces an about additive or a superadditive effect, h) the co-administration of the bispecific anti-ErbB2/anti-ErbB3 antibody with a receptor tyrosine kinase inhibitor (e.g., lapatinib) produces about a substantially additive or a superadditive effect. i) the bispecific anti-ErbB2/anti-ErbB3 antibody is the antibody comprising SEQ ID NO:1 and is administered in accordance with any of the regimens (e.g., modes, dosages, dosing intervals, loading and maintenance doses and dosing schemes) described in Examples 12 and 13, below, j) the lapatinib is administered in accordance with any of the regimens (e.g., modes, dosages, dosing intervals, loading and maintenance doses and dosing schemes) described in Example 16, below.

### BRIEF DESCRIPTION OF THE DRAWINGS

FIG. 1 is a graph showing that the combination of MM-111 and tamoxifen inhibits tumor growth in vivo better than either MM-111 or tamoxifen does alone. The x-axis shows time post tumor implant in days and the y-axis shows tumor volume in mm³. Mice were treated with inhibitors beginning on day 7 post BT474-M3 cell implant.

FIG. 2 is seven graphs showing that MM-111 combines positively with anti-estrogen drugs in inhibiting estrogen-stimulated spheroid growth in vitro. FIG. 2a shows the effect of MM-111, tamoxifen (4-hydroxytamoxifen or 4OHT), or MM-111 and tamoxifen on in vitro spheroid growth. FIG. 2b shows the effect of trastuzumab, tamoxifen, or trastuzumab and tamoxifen. FIG. 2c shows the effect of MM-111, fulvestrant (FVT), or MM-111 and fulvestrant. FIG. 2d shows the effect of trastuzumab, fulvestrant, or trastuzumab and fulvestrant. FIG. 2e shows the effect of MM-111, trastuzumab, or MM-111 and trastuzumab. FIG. 2f shows the effect of MM-111, trastuzumab, and tamoxifen combined compared to that of any of the double combinations. FIG. 2g shows the effect of MM-111, trastuzumab, and fulvestrant combined compared to that of any of the double combinations. The x-axes are a log scale of each drug concentration for each experimental condition in nM and the y axis is spheroid size as % of control spheroid size.

FIG. 3 is seven graphs showing that MM-111 combines positively with anti-estrogen drugs in inhibiting heregulin (HRG)-stimulated spheroid growth in vitro. FIG. 3a shows the effect of MM-111, tamoxifen (4-hydroxytamoxifen or 4OHT), or MM-111 and tamoxifen. FIG. 3b shows the effect of trastuzumab, tamoxifen, or trastuzumab and tamoxifen. FIG. 3c shows the effect of MM-111, fulvestrant (FVT), or MM-111 and fulvestrant. FIG. 3d shows the effect of trastuzumab, fulvestrant, or trastuzumab and fulvestrant. FIG. 3e shows the effect of MM-111, trastuzumab, or MM-111 and trastuzumab. FIG. 3f shows the effect of MM-111, trastuzumab, and tamoxifen combined compared to that of any of the double combinations. FIG. 3g shows the effect of MM-111, trastuzumab, and fulvestrant combined compared to that of any of the double combinations. The x-axes are a log scale of each drug concentration for each experimental condition in nM and the y axis is spheroid size as % of control spheroid size.

FIG. 4 is seven graphs showing that MM-111 combines positively with anti-estrogen drugs in inhibiting dual ligand (estrogen and heregulin)-stimulated spheroid growth in vitro. FIG. 4a shows the effect of MM-111, tamoxifen, or MM-111 and tamoxifen. FIG. 4b shows the effect of trastuzumab, tamoxifen, or trastuzumab and tamoxifen. FIG. 4c shows the effect of MM-111, fulvestrant (FVT), or MM-111 and fulvestrant. FIG. 4d shows the effect of trastuzumab, fulvestrant, or trastuzumab and fulvestrant. FIG. 4e shows the effect of MM-111, trastuzumab, or MM-111 and trastuzumab. FIG. 4f shows the effect of MM-111, trastuzumab, and tamoxifen combined compared to that of any of the double combinations. FIG. 4g shows the effect of MM-111, trastuzumab, and fulvestrant combined compared to that of any of the double combinations. The x-axes are a log scale of each drug concentration for each experimental condition in nM and the y axis is spheroid size as % of control spheroid size.

FIG. 5 is a graph summarizing the effect of MM-111, trastuzumab, and tamoxifen combined compared to that of any of the double combinations or MM-111, trastuzumab, and fulvestrant combined compared to that of any of the double combinations at inhibiting single ligand (estrogen or heregulin) or dual-ligand (estrogen and heregulin)-stimulated spheroid growth in vitro. The y-axis is % inhibition of spheroid size normalized to stimulated control.

FIG. 6 is a graph showing that the combination of MM-111 and lapatinib inhibits tumor growth in vivo. The x-axis shows the time post tumor implant in days and the y-axis shows tumor volume in mm³. Mice were treated with inhibitors on day 7 post tumor implant.

FIG. 7 evaluates the ability of lapatinib to inhibit ErbB3 and AKT activation in heregulin-stimulated cells. 7a is a graph comparing computer-generated dose-response curves to experimental results in heregulin-stimulated BT474-M3 cells. 7b is a graph showing lapatinib inhibition (IC50) of ErbB3 and AKT activation in heregulin-stimulated and unstimulated cells following a 1-hour incubation with inhibitor.

FIG. 8 is a series of graphs showing MM-111 or lapatinib inhibition of ErbB3 (8a) or AKT (8b) activation in heregulin-stimulated cells incubated with inhibitor for 15 minutes, 1 hour, 4 hours, and 24 hours. FIG. 8c shows a comparison of IC50 for MM-111 and lapatinib at 1 hour and 24 hours for both BT474M3 cells and ZR75-30 cells.

FIG. 9 is a graph showing the effect of MM-111 and lapatinib combination treatment on AKT activation in heregulin-stimulated BT474-M3 cells.

FIG. 10 is a graph showing the effect of lapatinib on cell viability as a measure of proliferation of unstimulated and heregulin-stimulated BT474-M3 cells.

FIG. 11 is a graph showing the effect of MM-111, lapatinib, or the combination on BT474-M3 cell apoptosis. The number of dead cells, cells in late apoptosis, early apoptosis, and live cells was quantitated.

FIG. 12 is three graphs showing that MM-111 combines positively with anti-estrogen drugs and lapatinib in inhibiting dual ligand (estrogen (E2) and heregulin (HRG))-stimulated spheroid growth in vitro. FIG. 12a shows the effect of lapatinib alone or the combination of lapatinib and fulvestrant (FVT). FIG. 12b shows the effect of lapatinib alone or the combination of lapatinib and MM-111. FIG. 12c shows the effect of lapatinib alone, the combination of MM-111 and fulvestrant, or the triple combination of MM-111, FVT, and lapatinib. Lapatinib is given in 3.3, 10, or 30 nM doses. The x-axes are a log scale of each of MM-111 and/or FVT concentration in nM and the y axis is spheroid size as % of control (FBS alone) spheroid size.

FIG. 13 is four graphs showing the MM-111 combines positively with the aromatase inhibitor letrozole and the tyrosine kinase inhibitor lapatinib in heregulin (HRG) and androstenedione (A4)-stimulated BT474-M3-Aro cells that stably express human aromatase, which converts androstenedione to estrogen. FIG. 13a shows the effect of letrozole, MM-111, or the combination of letrozole and MM-111. FIG. 13b shows the effect of lapatinib, MM-111 or the combination of lapatinib and MM-111. FIG. 13c shows the effect of lapatinib, letrozole, or the combination of lapatinib and letrozole. FIG. 13d shows the effect of the dual combinations of MM-111 and letrozole, MM-111 and lapatinib, lapatinib and letrozole, and the triple combination of MM-111, lapatinib and letrozole. The x-axes are a log scale of MM-111 concentration in nM. The drug concentrations are a ratio of 10:20:1 MM-111 to letrozole to lapatinib. The y axis is spheroid size as % of control spheroid size.

### DETAILED DESCRIPTION

As herein provided, bispecific anti-ErbB2/anti-ErbB3 antibodies (e.g., MM-111) are co-administered with one or more additional therapeutic agents (e.g. an aromatase inhibitor or tyrosine kinase inhibitor), to provide effective treatment to human patients having a cancer.

The term “anti-ErbB3 agent” refers to any therapeutic agent that binds to ErbB3 or binds to an ErbB3-specific ligand or blocks the expression of ErbB3, and thereby inhibits the activity of cellular signaling mediated by ErbB3. Non-limiting examples of types of anti-ErbB3 agents include antibodies, bispecific antibodies, ligand analogs, soluble forms of ErbB3 or the ErbB3 ectodomain, ErbB3 specific RNAi molecules, and similar biologic agents.

The term “antibody” describes a polypeptide comprising at least one antibody-derived antigen binding site (e.g., V_{H}/V_{L} region or Fv, or complementarity determining region—CDR) that specifically binds to a specific antigen, e.g., ErbB3. “Antibodies” include whole antibodies and any antigen binding fragment, e.g., Fab or Fv, or a single chain fragment (e.g., scFv), as well as bispecific antibodies and similar engineered variants, human antibodies, humanized antibodies, chimeric antibodies Fabs, Fab′2s, ScFvs, SMIPs, Affibodies®, nanobodies, or a domain antibodies, and may be of any of the following isotypes: IgG1, IgG2, IgG3, IgG4, IgM, IgA1, IgA2, IgAsec, IgD, and IgE. The antibody may be a naturally occurring antibody or may be an antibody that has been altered (e.g., by mutation, deletion, substitution, conjugation to a non-antibody moiety). For example, an antibody may include one or more variant amino acids (compared to a naturally occurring antibody) which change a property (e.g., a functional property) of the antibody. For example, numerous such alterations are known in the art which affect, e.g., half-life, effector function, and/or immune responses to the antibody in a patient. The term “antibody” thus includes whole antibodies and any antigen binding fragment (i.e., “antigen-binding portion,” e.g., Fabs) or single chains thereof (e.g., scFvs) as well as bispecific antibodies and similar engineered variants, provided that they retain the binding specificity of an antibody.

An “anti-ErbB3 antibody” is an antibody that immunospecifically binds to the ectodomain of ErbB3 and an “anti-ErbB2 antibody” is an antibody that immunospecifically binds to the ectodomain of ErbB2. The antibody may be an isolated antibody. Such binding to ErbB3 or ErB2 exhibits a Kd with a value of no greater than 50 nM as measured by a surface plasmon resonance assay or a cell binding assay. Exemplary anti-ErbB3 antibodies inhibit EGF-like ligand mediated phosphorylation of ErbB3, e.g., anti-ErbB2 antibodies that inhibit the binding of heregulin to ErbB2/ErbB3 heterodimers. EGF-like ligands include EGF, TGFα, betacellulin, heparin-binding epidermal growth factor, biregulin, epigen, epiregulin, and amphiregulin, which typically bind to ErbB1 and induce heterodimerization of ErbB1 with ErbB3.

The term “bispecific antibody” as used herein refers to a protein comprising two antigen-binding sites, a first binding site exhibiting immunospecific binding to a first antigen or epitope and a second binding site exhibiting immunospecific binding to a second antigen or epitope distinct from the first. An “anti-ErbB2/anti-ErbB3 bispecific antibody” is an antibody that comprises two binding sites, one that immunospecifically binds to the ectodomain of ErbB3 and another that immunospecifically binds to the ectodomain of ErbB2. Preferably, a bispecific ErbB3, ErbB2 antibody is the antibody comprising SEQ ID NO:1.

An “anti-estrogen agent” as used herein refers to an agent that prevents or reduces production of estrogen or prevents or reduces signaling mediated by estrogen receptors. Anti-estrogen agents include but are not limited to estrogen receptor antagonists and aromatase inhibitors. Estrogen receptor antagonists include but are not limited to raloxifene, fulvestrant, tamoxifen, afimoxifene (4-hydoroxytamoxifen), arzoxifene, toremifene, and lasofoxone. Preferably, the estrogen receptor antagonist is tamoxifen or fulvestrant. Aromatase inhibitors work by blocking the synthesis of estrogen in an animal (e.g., a mouse or a human). This lowers estrogen levels in the animal and thereby inhibits the growth of estrogen-driven cancers. Examples of aromatase inhibitors include but are not limited to exemestane, anastrozole, letrozole, aminoglutethimide, testolactone, vorozole, formestane, and fadrozole. Preferably, the aromatase inhibitor is exemestane or letrozole.

By “cancer” is meant any condition characterized by abnormal, unregulated, malignant cell growth.

By “malignant tumor” is meant any cancer that takes the form of a tumor.

The term “effective amount” refers to an amount of a drug effective to achieve a desired effect, e.g., to ameliorate disease in a subject. Where the disease is a cancer, the effective amount of the drug may inhibit (e.g., slow to some extent, inhibit or stop) one or more of the following characteristics: cancer cell growth, cancer cell proliferation, cancer cell motility, cancer cell infiltration into peripheral organs, tumor metastasis, and tumor growth. Where the disease is a cancer, the effective amount of the drug may alternately do one or more of the following when administered to a subject: slow or stop tumor growth, reduce tumor size (e.g., volume or mass); relieve to some extent one or more of the symptoms associated with the cancer, extend progression free survival, result in an objective response (including a partial response or a complete response), and increase overall survival time. To the extent the drug may prevent growth and/or kill existing cancer cells, it is cytostatic and/or cytotoxic.

A “mouse effective amount” refers to an amount of a drug effective to achieve a desired effect when the subject is a mouse.

A “human effective amount” refers to an amount of a drug effective to achieve a desired effect when the subject is a human patient.

The terms “combination therapy,” “concomitant use,” “co-administration,” co-administering,” “co-administered,” and the like, refer to the administration of at least two therapeutic agents to a subject either simultaneously or within a time period during which the effects of the earlier-administered therapeutic agent are still operative in the subject when a later-administered therapeutic agent is administered.

A “receptor tyrosine kinase inhibitor” as used herein refers to a member of a class of drugs that specifically inhibit receptor tyrosine kinases and thus reduce or eliminate the activation of various signal transduction pathways. Receptor tyrosine kinase inhibitors useful for the treatment of cancer as disclosed herein include but are not limited to the small molecule inhibitors erlotinib, afatinib, dasatinib, gefitinib, imatinib, pazopinib, lapatinib, sunitinib, nilotinib and sorafenib. Receptor tyrosine kinase inhibitors also include antibody-based therapeutics such as cetuximab, panitumumab, zalutumumab, nimotuzumab, and matuzumab). Preferably, the receptor tyrosine kinase inhibitor is lapatinib.

“Dosage” or “dosing regimen” refers to parameters for administering a drug in defined quantities per unit time (e.g., per hour, per day, per week, per month, etc.) to a patient. Such parameters include, e.g., the size of each dose. Such parameters also include the configuration of each dose, which may be administered as one or more units, e.g., taken at a single administration, e.g., orally (e.g., as one, two, three or more pills, capsules, etc.) or injected (e.g., as a bolus). Dosage sizes may also relate to doses that are administered continuously (e.g., as an intravenous infusion over a period of minutes or hours). Such parameters further include frequency of administration of separate doses, which frequency may change over time. A “dosing cycle” or “dosing interval” is the period of time that comprises one cycle of treatment (e.g., 21 days or 28 days) for a dosing regimen.

“Dose” refers to an amount of a drug given in a single administration.

Preferred cancer cells of cell lines are cells of ErbB2 expressing cell lines such as ErbB2 overexpressing cell lines, e.g., BT474-M3 (ATCC® #CRL-HTB-20™, derived from breast ductal carcinoma cells), BT474-M3-Aro (BT474-M3 cells that stably express human aromatase), ZR75-30 (ATCC® #CRL-1504™, derived from breast ductal carcinoma cells), SKOV-3 (ATCC® #HTB-77™, derived from metastatic ovarian adenocarcinoma cells), MCF7 (ATCC® #HTB-22™) clone 18, MDA-MB-453 (ATCC® #HTB-131™, derived from breast carcinoma cells), SK-BR-3 (ATCC® #HTB-30™, derived from breast adenocarcinoma cells), and NCI-N87 (ATCC® #CRL-5822™, derived from gastric carcinoma cells).

Cancers may include, for example, solid tumors such as: sarcomas (e.g., clear cell sarcoma), carcinomas (e.g., renal cell carcinoma), and lymphomas; tumors of the breast, colon, rectum, lung, oropharynx, hypopharynx, esophagus, stomach, pancreas, liver, bilecyst, bile duct, small intestine, urinary system (including the kidney, bladder, and epithelium of the urinary tract), female genital system (including the uterine neck, uterus, ovary, chorioma, and gestational trophoblast), male genital system (including the prostate, seminal vesicle, and testicles), endocrine glands (including the thyroid gland, adrenal gland, and pituitary body), skin (including angioma, melanoma, sarcoma originating from bone or soft tissue, and Kaposi's sarcoma), brain and meninges (including astrocytoma, neuroastrocytoma, spongioblastoma, retinoblastoma, neuroma, neuroblastoma, neurinoma and neuroblastoma), nerves, and eyes.

A cancer may be an estrogen receptor positive (ER+) cancer. Such cancers exemplify candidates for therapy regimens that include anti-estrogen agents. Such cancers may include but are not limited to certain breast, ovarian, uterine, endometrial, lung, bone, brain, bladder, liver and urogenital cancers.

A cancer may be an ErbB2 gene-amplified cancer and/or an ErbB2-expressing or overexpressing cancer. ErbB2, also known as HER2 or Neu, is a cell surface transmembrane receptor protein that generates intracellular signals (e.g., upon ligand activation) via its intracellular tyrosine kinase activity. In excess, such signals can promote oncogenesis e.g., by triggering cell division. The ErbB2 gene is amplified and/or overexpressed in many types of human malignancies, including but not limited to breast, ovarian, endometrial, pancreatic, colorectal, prostate, salivary gland, kidney, and lung. ErbB2 overexpressing cancers are designated a HER2⁺⁺⁺ or HER2⁺⁺ depending on the level of ErbB2 overexpression, with HER2⁺⁺⁺ indicating the highest levels of HER2 expression. HER2⁺⁺⁺ and HER2⁻⁺ status are typically determined by an immunoassay such as immunohistochemistry, e.g., Herceptest®. ErbB2 gene amplification is may be determined by, e.g., FISH (fluorescence in situ hybridization), with HER2-amplified cancer cells being those that have more than two HER2 gene copies being HER2-amplified, and cells and/or tumors comprising HER2-amplified cancer cells being referred to as “FISH positive.”

A number of bispecific anti-ErbB2, antiErbB3 antibodies that are scFv HSA conjugates are described in co-pending US patent publication No. 2011-0059076, and PCT publication Nos. WO2009/126920 and WO 2010/059315, each of which is incorporated herein by reference in its entirety and each of which discloses MM-111 (also referred to as B2B3-1) and other bispecific anti-ErbB2/antiErbB3 antibodies that are scFv HSA conjugates and that are suitable for use in the methods and compositions provided herein, including the components of A5-HSA-ML3.9, ML3.9-HSA-A5, A5-HSA-B1D2, B1D2-HSA-A5, B12-HSA-B1D2, B1D2-HSA-B12, A5-HSA-F5B6H2, F5B6H2-HSA-A5, H3-HSA-F5B6H2, F5B6H2-HSA-H3, F4-HSA-F5B6H2, F5B6H2-HSA-F4, B1D2-HSA-H3, and H3-HSA-B1D2. Other suitable bispecific anti-ErbB2/antiErbB3 antibodies are disclosed and claimed in U.S. Pat. Nos. 7,332,580 and 7,332,585, which are incorporated herein by reference. MM-111 is currently undergoing clinical trials, including an open-label Phase 1/2 and pharmacologic study of MM-111 in patients with advanced, refractory HER2 positive cancers, an open-label Phase 1/2 trial of MM-111 in combination with trastuzumab (Herceptin®) in patients with advanced HER2 positive breast cancer, and an open label, Phase 1/2 and pharmacologic study of MM-111 with three different combination treatments: MM-111 in combination with cisplatin, capecitabine, and trastuzumab, MM-111 in combination with lapatinib and trastuzumab, and MM-111 in combination with paclitaxel and trastuzumab.

A bispecific anti-ErbB2/anti-ErbB3 antibody (e.g., MM-111) can be co-administered with other therapeutic agents, (e.g., an anti-estrogen receptor agent or a receptor tyrosine kinase inhibitor) prior to (e.g., neoadjuvant therapy), concurrent with, or following (e.g., adjuvant therapy) radiotherapy of, or surgical intervention to remove, a malignant tumor.

Additional therapeutic agents suitable for combination with anti-ErbB2/anti-ErbB3 antibodies may further include: 1) monoclonal antibody EGFR inhibitors (e.g. cetuximab, panitumumab, zalutumumab, nimotuzumab, and matuzumab), additional small molecule tyrosine kinase inhibitors such as PKI-166, PD-158780, EKB-569, Tyrphostin AG 1478, and pan-HER kinase inhibitors (e.g. CI-1033 (PD 183805), AC480, HM781-36B, AZD8931 and PF299804); 2) microtubule stabilizing agents (e.g. laulimalide, epothilone A, epothilone B, discodermolide, eleutherobin, sarcodictyin A, sarcodictyin B, paclitaxel, nab-paclitaxel or docetaxel); antimetabolites such as 5-fluorouracil (5-FU) and capecitabine; and platinum-based therapeutics such as oxaliplatin, carboplatin and cisplatin. Additional examples of therapeutic agents suitable for combination with anti-ErbB2/anti-ErbB3 antibodies may be found in Table 5 and the Appendix below.

MM-111 is suitable for both large scale production and systemic therapy. MM-111 binds to ErbB2/ErbB3 heterodimers and forms a trimeric complex with ErbB2 and ErbB3, effectively inhibiting ErbB3 signaling. The antitumor activity of MM-111 requires the presence of both ErbB2 and ErbB3, but is particularly dependent on ErbB2 expression. The affinity of its ErbB2 antigen-binding site is about 30 times higher than the affinity of its ErbB3 antigen-binding site, but the ErbB2 antigen-binding site does not by itself inhibit ErbB2 activity when bound to ErbB2. The strong binding of MM-111 to ErbB2 places the ErbB3 antigen-binding site in close proximity to bound ErbB2/ErbB3 heterodimer, resulting in an avidity effect that potentiates the binding of the ErbB3 antigen-binding site to the heterodimer ErbB3, whereby a biological effect is produced. MM-111 is administered to human subjects (patients) at an interval measured in days, as a single loading dose of at least 20 mg/kg of MM-111 followed by at least seven day intervals (e.g., every 2 weeks) by at least one administration of a single maintenance dose of MM-111, where the maintenance dose is generally smaller than the loading dose, e.g., at least 5 mg/kg less than the loading dose.

### EXAMPLES

The following examples are provided by way of illustration only and not by way of limitation. Those of skill in the art will readily recognize a variety of non-critical parameters that could be changed or modified to yield essentially the same or similar results.

MM-111 in Combination with Anti-Estrogen Therapeutics

### Methods:

### Spheroid In Vitro Tumor Model Assay

BT474-M3 wild type cells (2000 cells/well) are plated in Ultra Low Cluster 96-well plate (Costar). After overnight incubation, indicated treatments are introduced to the plate. Cells are continued to culture for six days. Spheroids are then examined by Nikon microscope and analyzed by MetaMorph Image Analysis Software (Molecular Devices). The spheroid size from cells cultured in medium containing 10% FBS is set as control.

### Xenograft Model

BT474-M3 cells (2×10⁷ cells per mice) are inoculated subcutaneously into Nu/Nu immunodeficient mice, which are implanted with an estrogen pellet (0.72 mg; 60-day release) one day before the experiment. Tumors are measured after seven days and mice are randomized into four groups: those treated with placebo, MM-111 (60 mg/kg, Q7D), 4-hydroxytamoxifen (5 mg; 60-day release pellet), and combination of MM-111 and 4-hydroxytamoxifen, respectively. Tumors are measured every three days and the experiment is ended at day 32.

### Example 1

### MM-111 and Tamoxifen Combination Therapy Inhibits Tumor Growth In Vivo

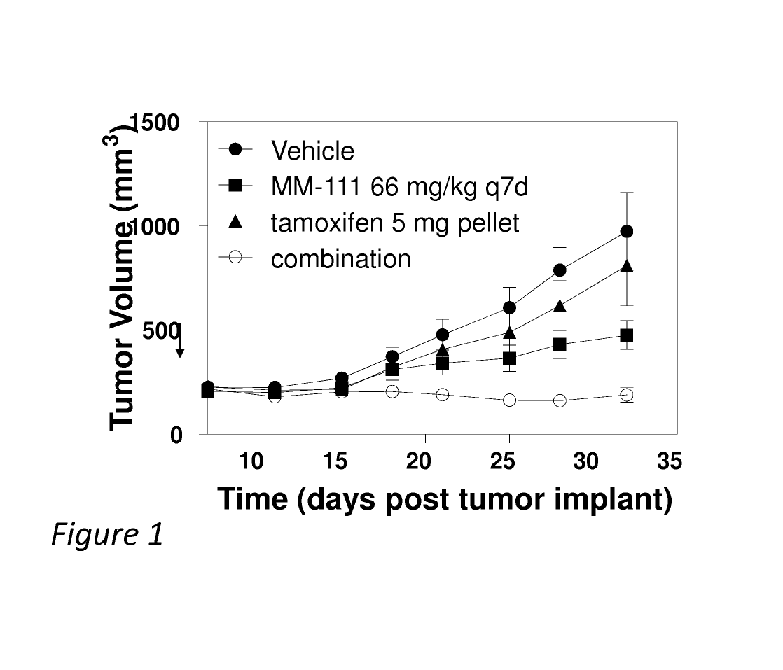

In order to compare the effect of MM-111 and tamoxifen combination therapy on tumor growth in vivo, estrogen stimulated mice were prepared in the xenograft model using the methods described above or minor variations thereof. Mice were inoculated with tumor forming BT474-M3 cells and on day 7 given a placebo (vehicle control), MM-111, tamoxifen, or a combination of MM-111 and tamoxifen and tumor growth was measured over time. As shown in FIG. 1, this in vivo BT474-M3 xenograft model showed resistance to tamoxifen treatment but when mice were given a combination of MM-111 and tamoxifen the combination treatment inhibited tumor growth to a significantly greater extent. Statistical significance (p<0.05) was observed for the combination group from day 28 onward when compared to vehicle control, from day 21 onward when compared to MM-111 and from day 25 onward when compared to tamoxifen.

### Example 2

### MM-111 Combines Positively with Anti-Estrogen Drugs in Inhibiting Estrogen-Stimulated Spheroid Growth

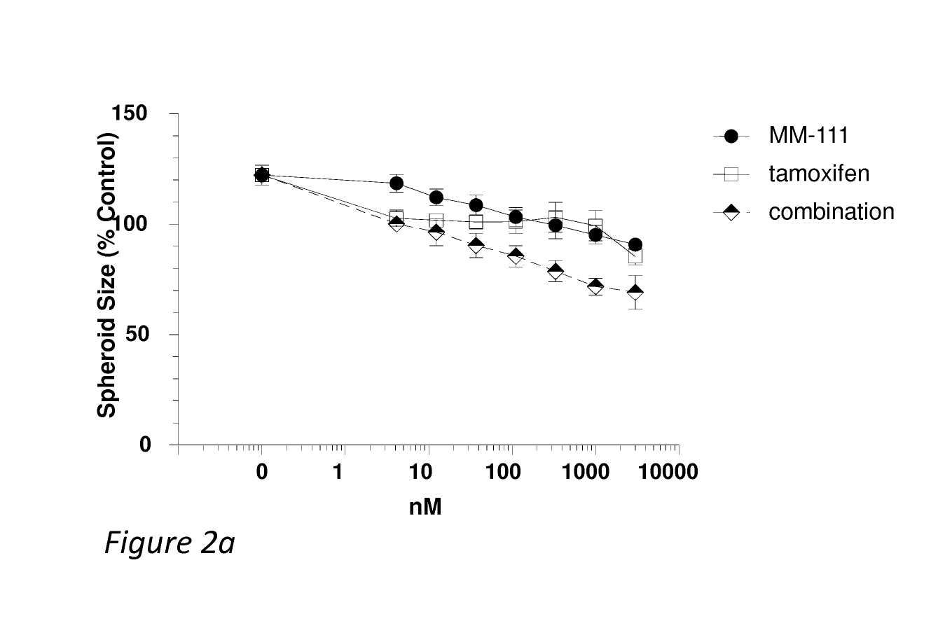

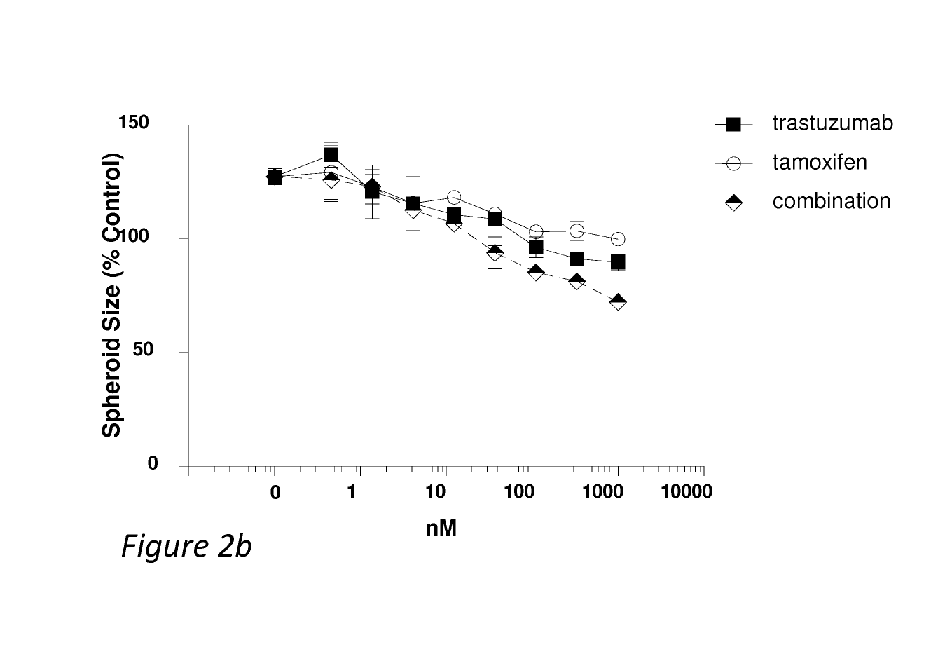

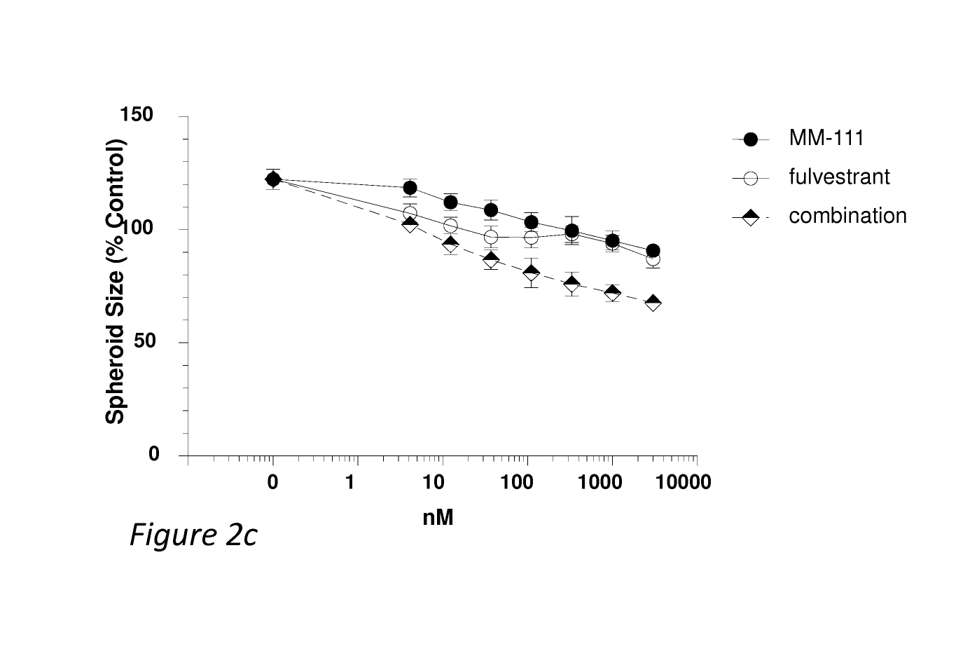

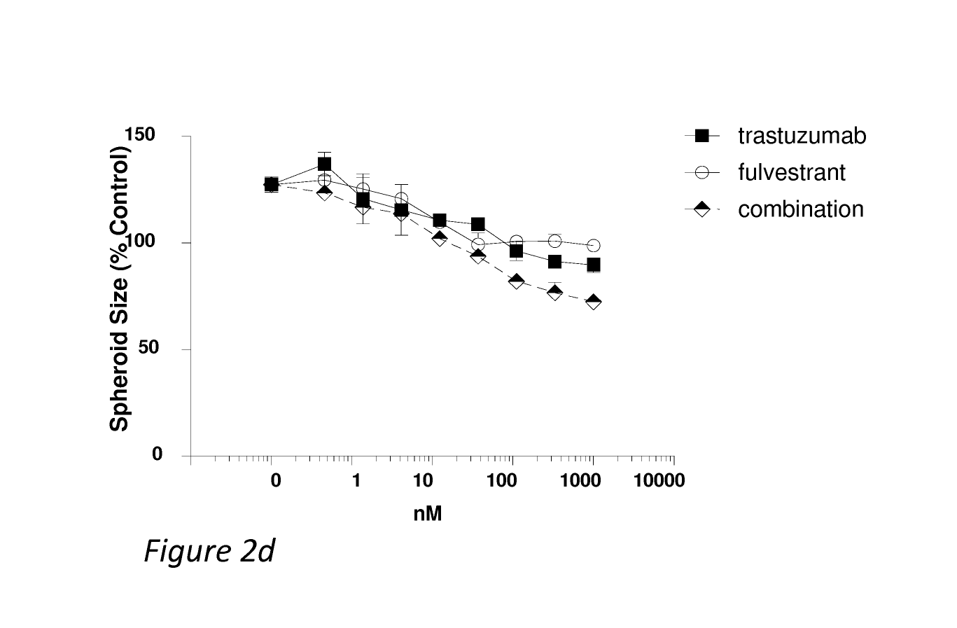

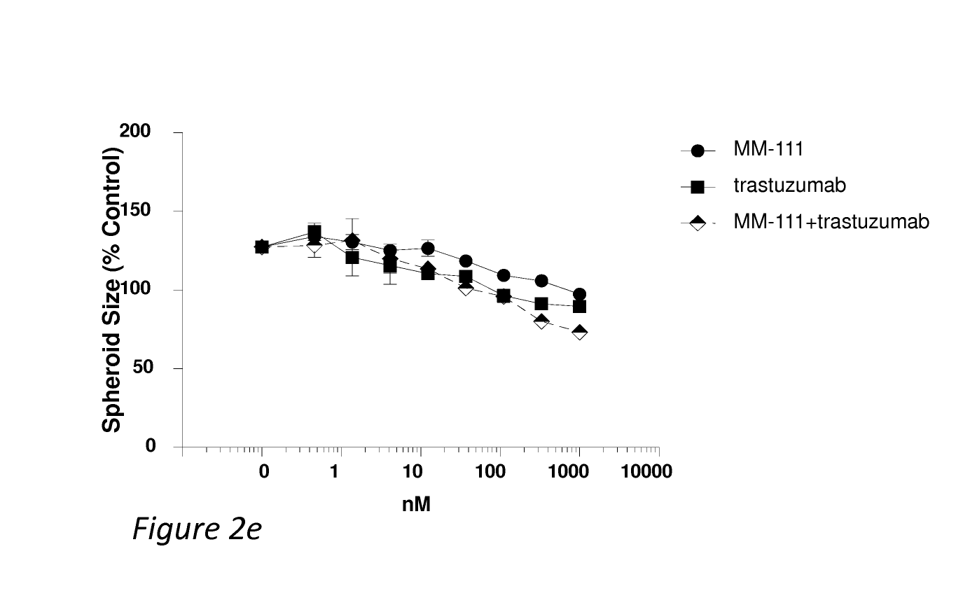

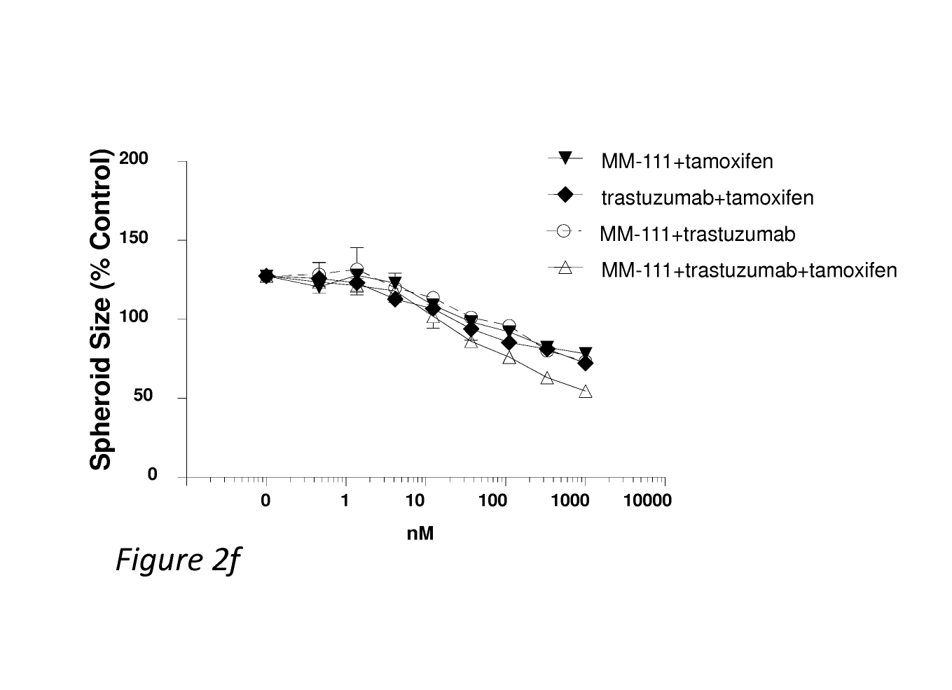

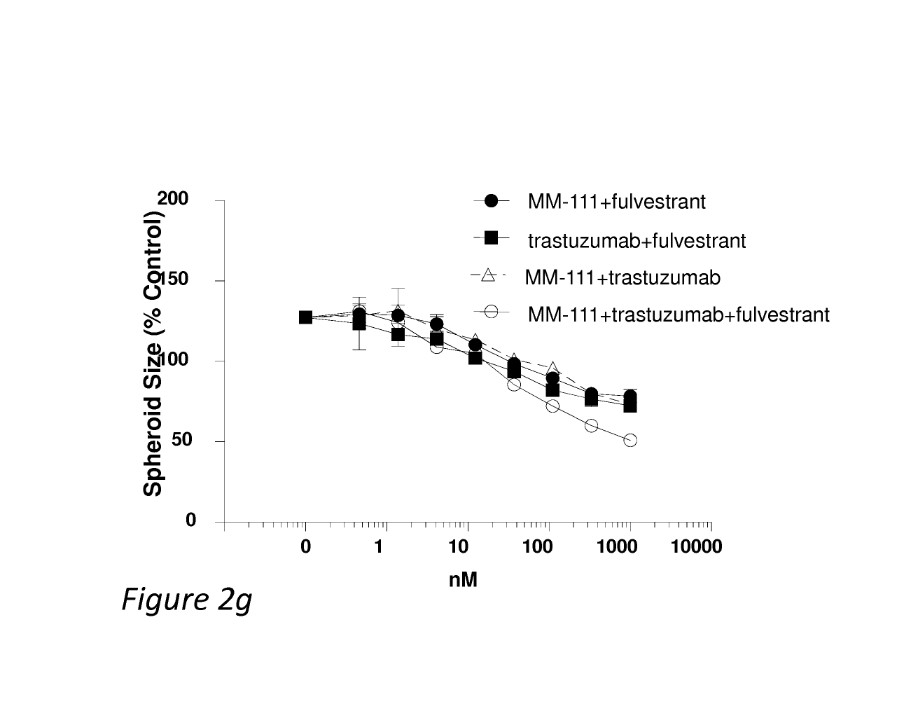

Multicellular spheroids are used to simulate the growth and microenvironmental conditions of tumors in vitro. To further investigate the ability of MM-111 to inhibit cell growth when in combination with anti-estrogen drugs, spheroids of BT474-M3 cells were prepared using the methods described above or minor variations thereof and treated with an ErbB2-binding therapeutic and/or an anti-estrogen therapeutic. Spheroids of estrogen-stimulated cells were treated with a dose range of MM-111, tamoxifen, or the combination of MM-111 and tamoxifen (FIG. 2a); trastuzumab, tamoxifen or the combination of trastuzumab and tamoxifen (FIG. 2b); MM-111, fulvestrant, or the combination of MM-111 and fulvestrant (FIG. 2c); trastuzumab, fulvestrant, or the combination of trastuzumab and fulvestrant (FIG. 2d); or MM-111, trastuzumab, or the combination of MM-111 and trastuzumab (FIG. 2e). When used as single agent alone, MM-111, trastuzumab, fulvestrant and tamoxifen showed inhibitory effects on spheroid growth in the estrogen-stimulated BT474-M3 spheroid assay. The combination of tamoxifen or fulvestrant with MM-111 (FIGS. 2a and 2c, respectively) or trastuzumab (FIGS. 2b and 2d, respectively) increased the degree of growth inhibition, as did the combination of MM-111 and trastuzumab (FIG. 2e). The inhibitory effects were increased still further when estrogen-stimulated spheroids were treated with the triple combination of MM-111, trastuzumab, and tamoxifen (FIG. 2f) or MM-111, trastuzumab, and fulvestrant (FIG. 2g) as compared to the double combinations of drugs.

### Example 3

### MM-111 Combines Positively with Anti-Estrogen Drugs in Inhibiting Heregulin-Stimulated Spheroid Growth

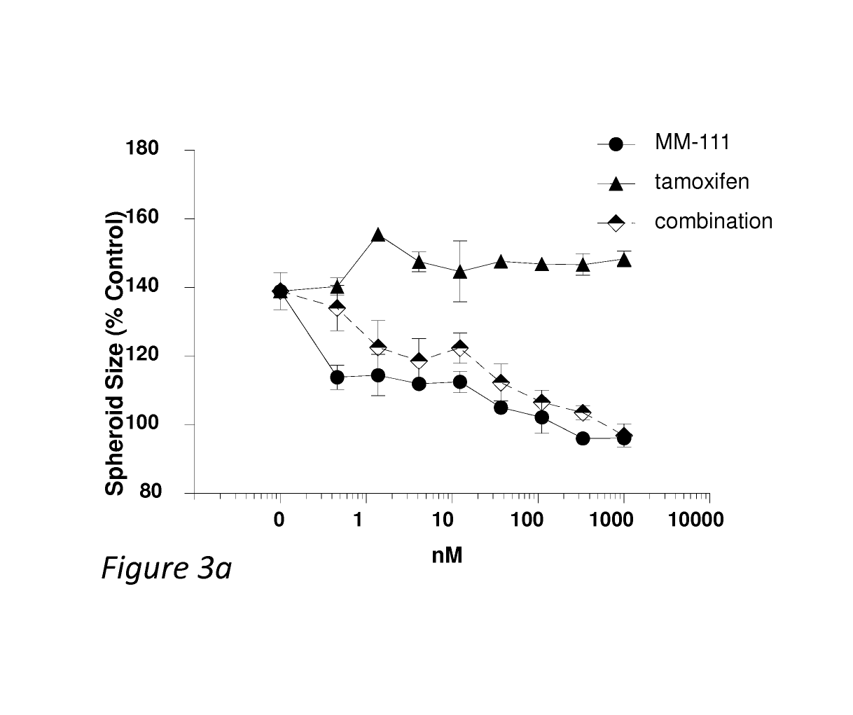

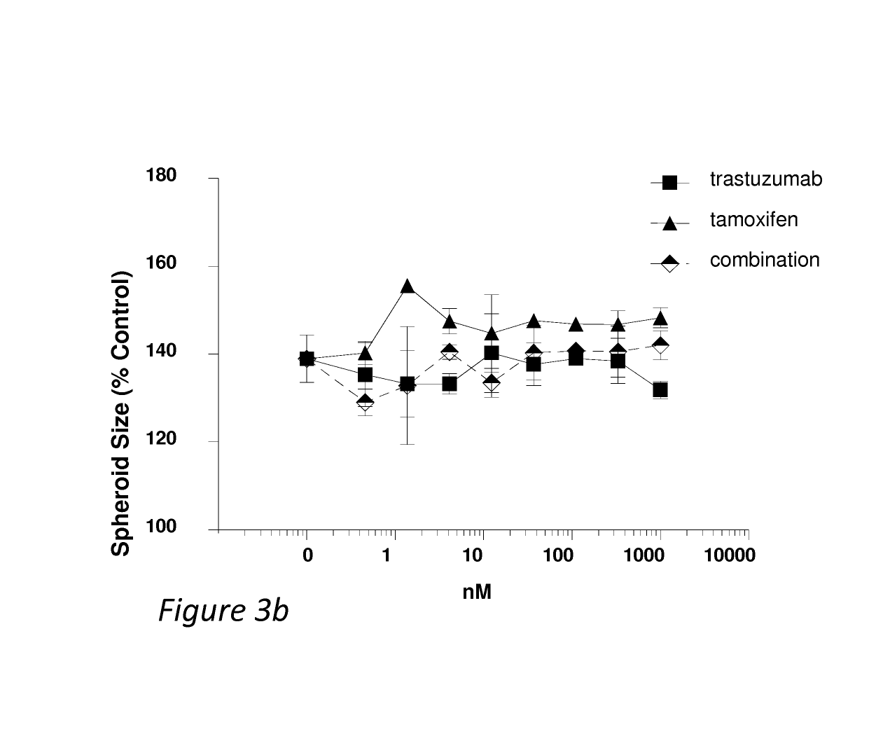

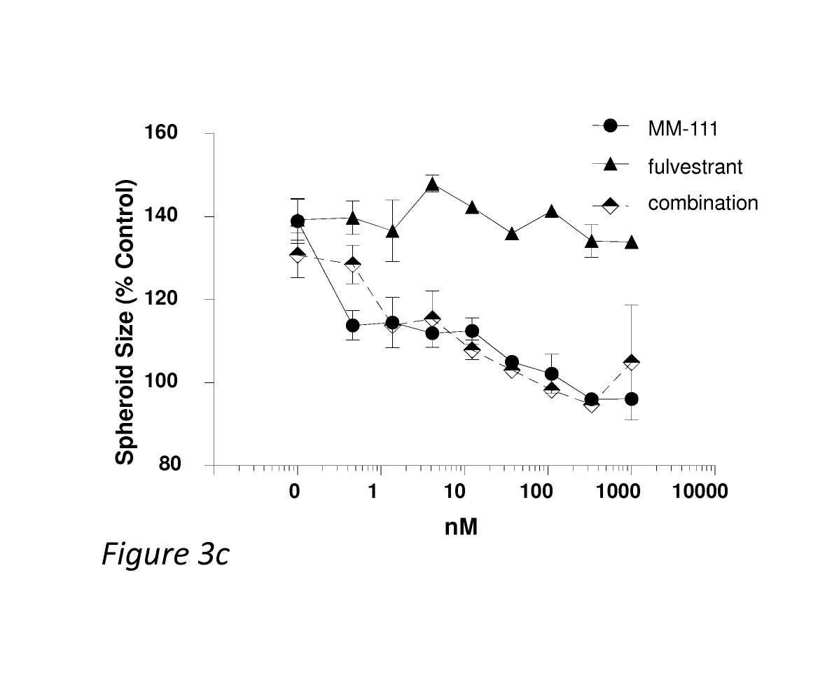

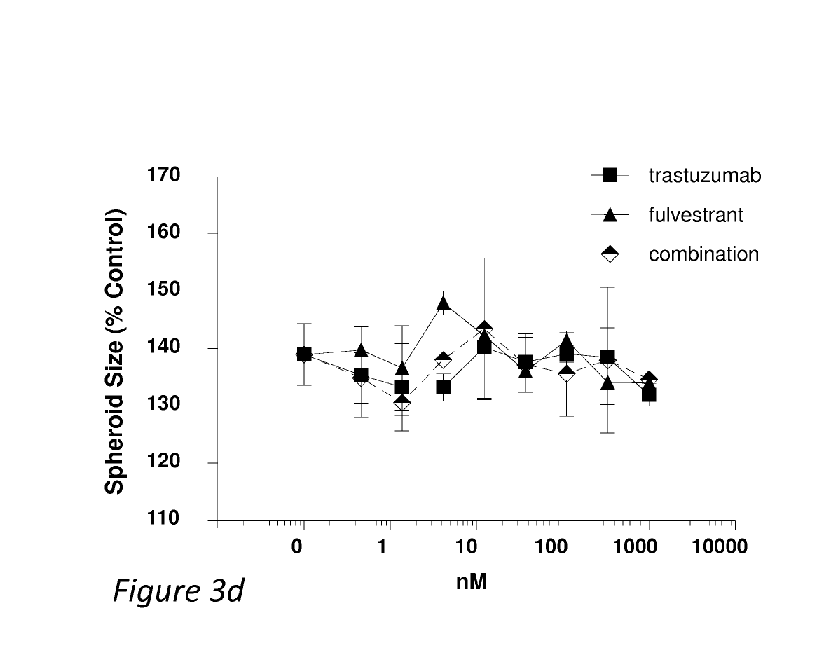

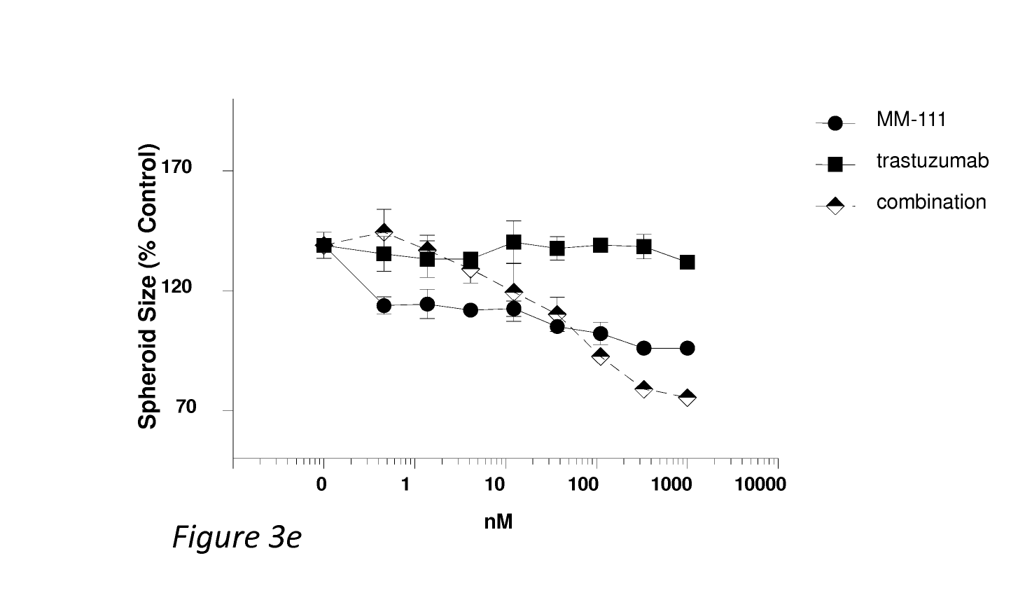

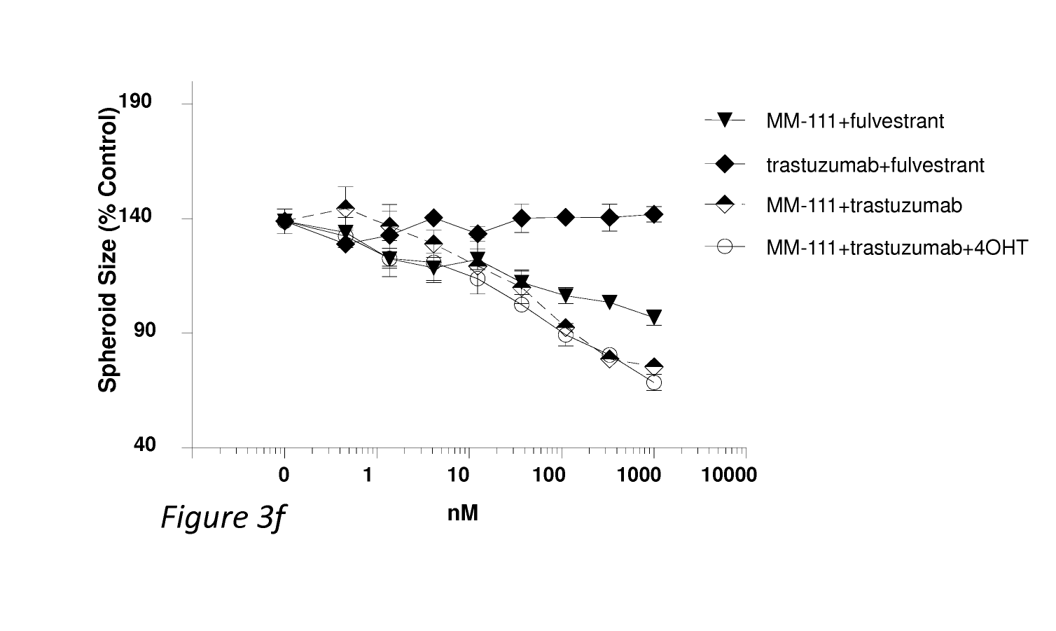

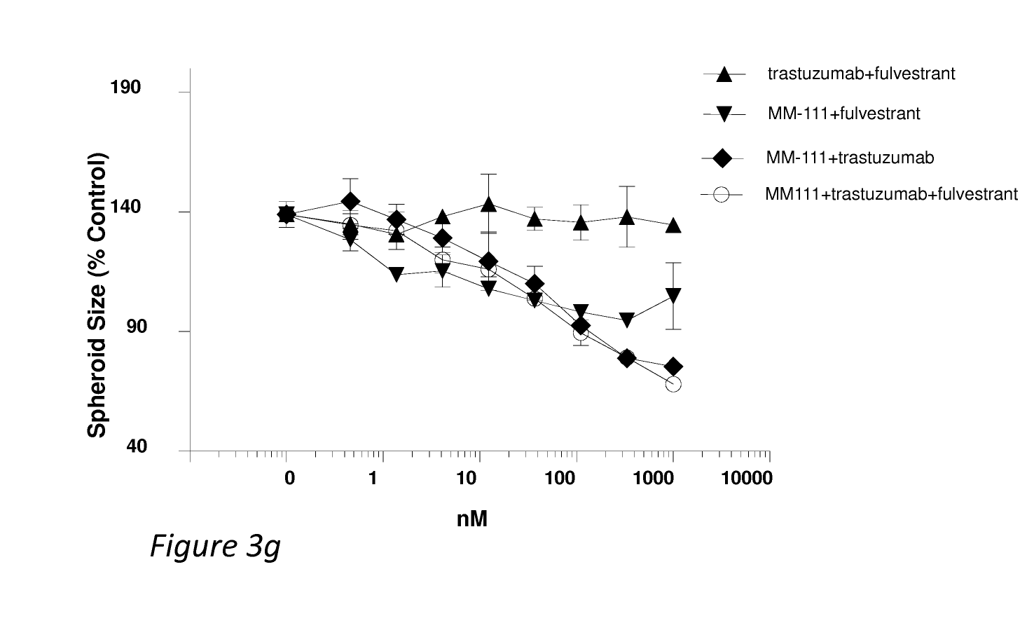

To further investigate the ability of MM-111 to inhibit cell growth when in combination with anti-estrogen drugs, spheroids of heregulin (HRG)-stimulated BT474-M3 cells were prepared using the methods described above or minor variations thereof and treated with a dose range of MM-111, tamoxifen, or the combination of MM-111 and tamoxifen (FIG. 3a); trastuzumab, tamoxifen or the combination of trastuzumab and tamoxifen (FIG. 3b); MM-111, fulvestrant, or the combination of MM-111 and fulvestrant (FIG. 3c); trastuzumab, fulvestrant, or the combination of trastuzumab and fulvestrant (FIG. 3d); or MM-111, trastuzumab, or the combination of MM-111 and trastuzumab (FIG. 3e). MM-111 inhibited heregulin-induced spheroid growth but tamoxifen (FIG. 3a), trastuzumab (FIG. 3b), and fulvestrant (FIG. 3c) did not inhibit heregulin stimulated spheroid growth. No significant combinational effect was observed when MM-111 was used with tamoxifen (FIG. 3a) or fulvestrant (FIG. 3c). The combination of trastuzumab and either tamoxifen (FIG. 3b) or fulvestrant (FIG. 3d) failed to show inhibitory activity significantly greater than either drug alone. As shown in FIG. 3e, MM-111 but not trastuzumab showed inhibitory activity in heregulin-stimulated spheroid growth. Improved inhibitory effects were observed when both drugs were combined. In comparison to the double combination of either MM-111 or trastuzumab with tamoxifen or fulvestrant, the triple combination of MM-111, trastuzumab and either tamoxifen (FIG. 3f) or fulvestrant (FIG. 3g) showed similar inhibitory effects as those of MM-111 and trastuzumab in combination (FIG. 3e) on heregulin-stimulated spheroid growth.

### Example 4

### MM-111 Combines Positively with Anti-Estrogen Drugs in Inhibiting Dual Ligand (Estrogen and Heregulin)-Stimulated Spheroid Growth

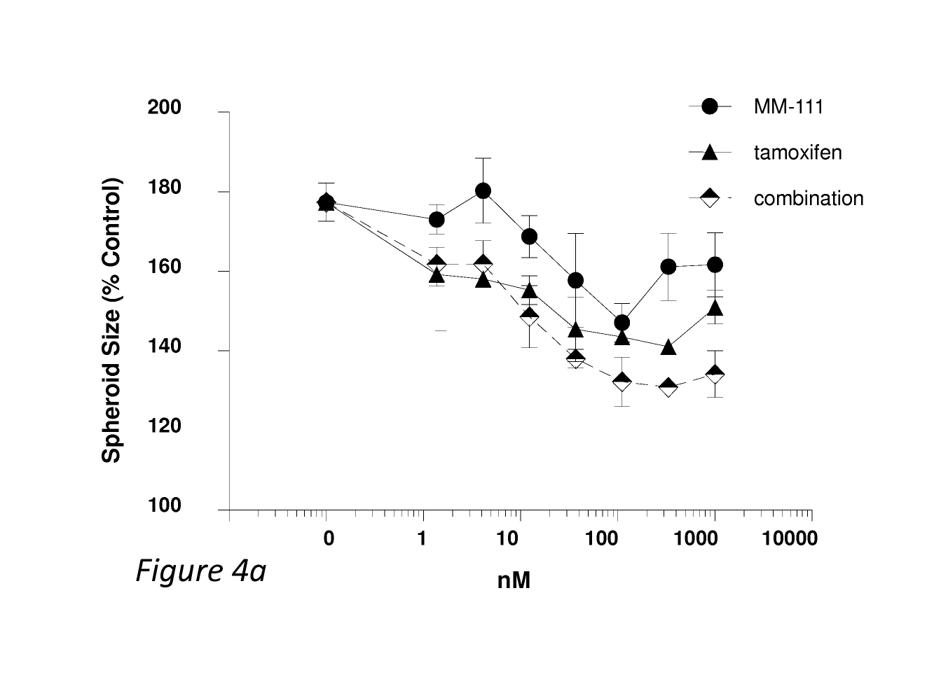

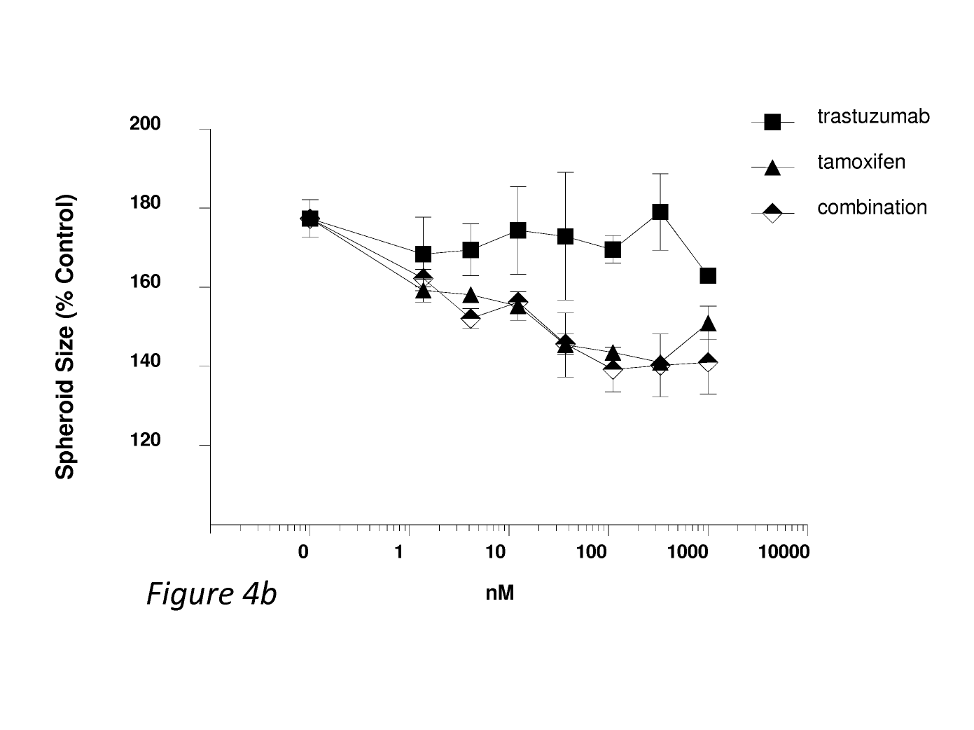

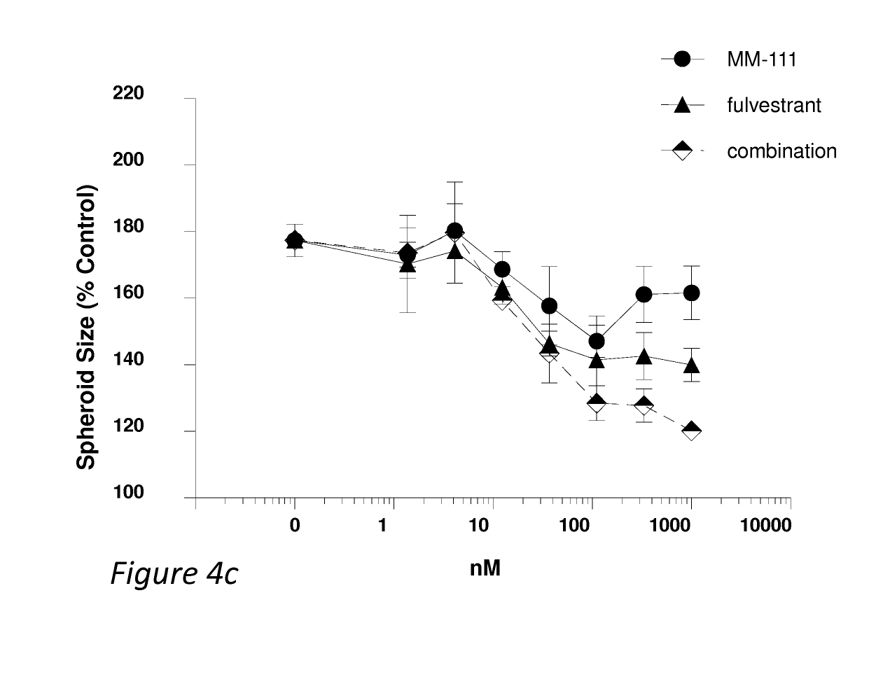

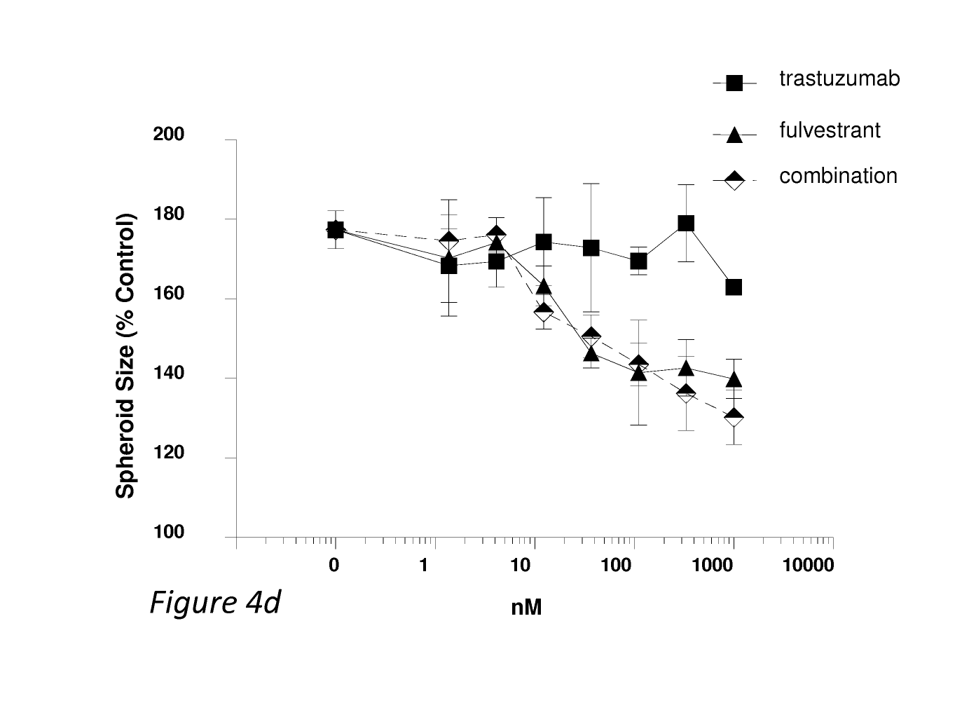

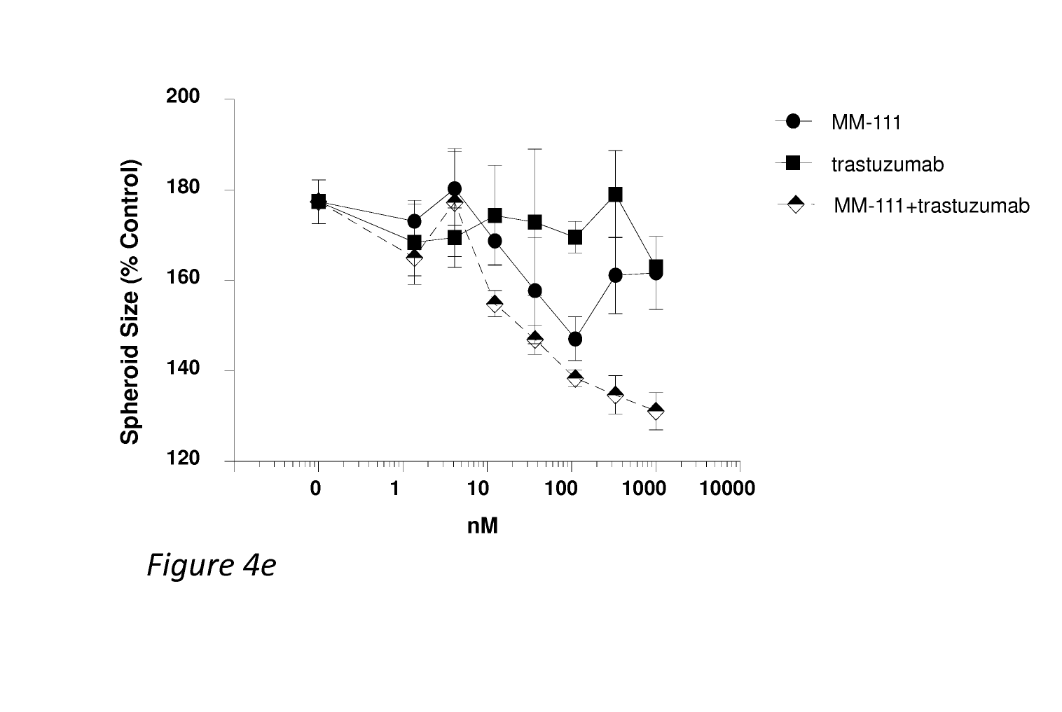

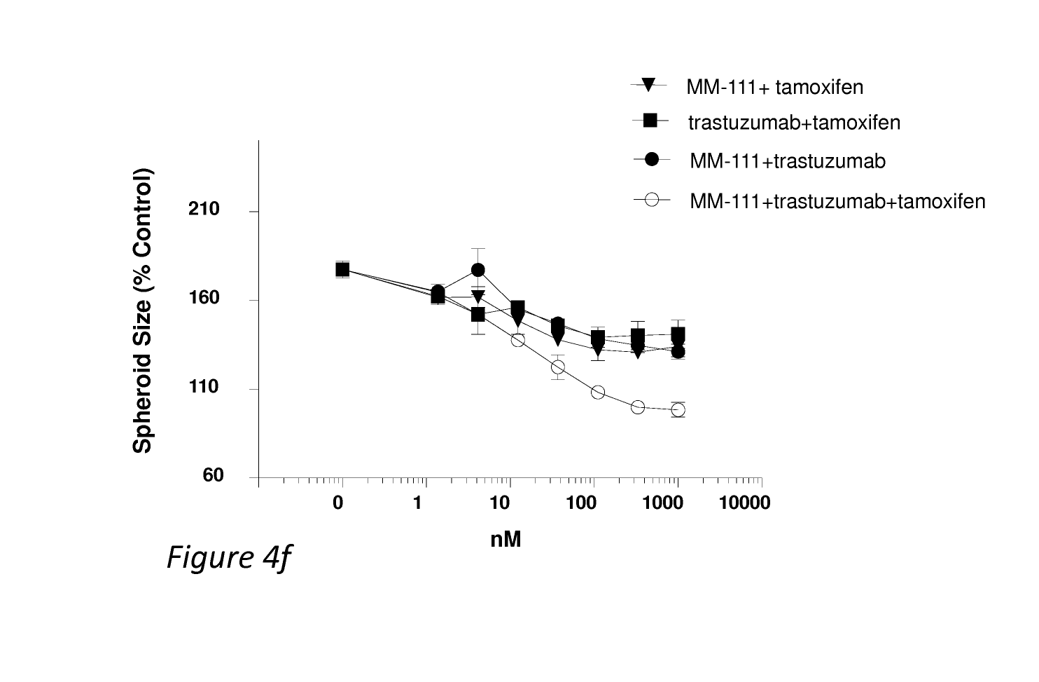

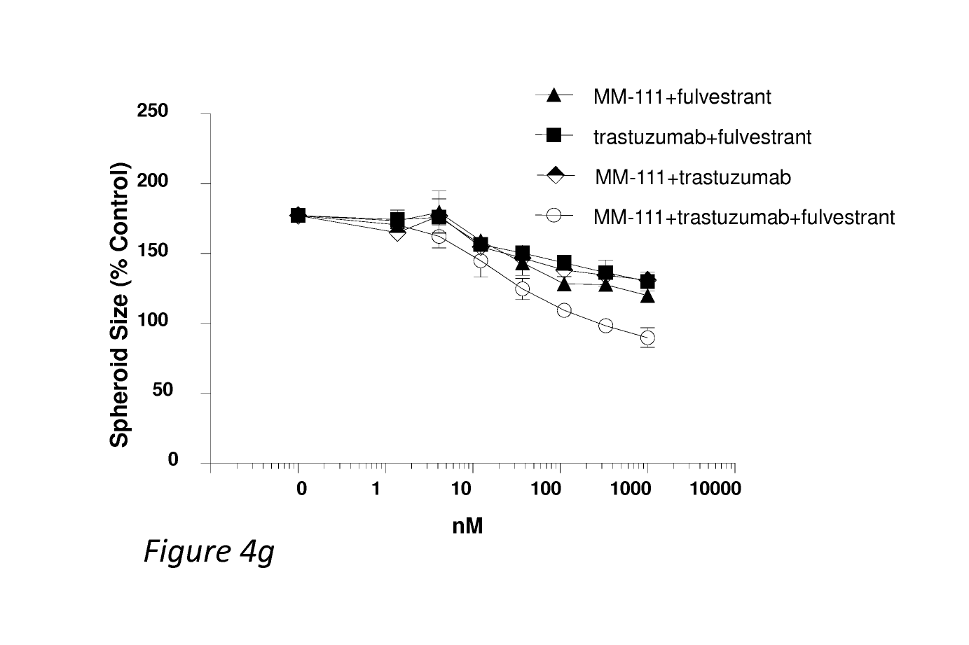

Dual ligand (estrogen and heregulin) stimulated spheroids were treated with a dose range of tamoxifen, MM-111 or the combination of MM-111 and tamoxifen (FIG. 4a) or trastuzumab, tamoxifen or the combination of trastuzumab and tamoxifen (FIG. 4b). While MM-111 and trastuzumab each inhibited spheroid growth (FIG. 4a) the combination of MM-111 and tamoxifen showed greater inhibitory effects than either drug alone. In contrast, trastuzumab alone had no significant inhibitory effects and the combination of trastuzumab and tamoxifen showed similar effects to tamoxifen alone.

Dual ligand stimulated spheroids were then treated with a dose range of fulvestrant, MM-111 or the combination of MM-111 and fulvestrant (FIG. 4c) or fulvestrant, trastuzumab, or a combination of fulvestrant or trastuzumab (FIG. 4d). Again, while MM-111 and fulvestrant each separately inhibited spheroid growth the combination of MM-111 and fulvestrant showed greater inhibitory effects than either drug alone (FIG. 4c). Trastuzumab alone had no significant inhibitory effects and the combination of trastuzumab and fulvestrant showed similar effects to tamoxifen alone (FIG. 4d).

Dual ligand stimulated spheroids were then treated with MM-111, trastuzumab, or a combination of MM-111 and trastuzumab. MM-111 showed greater inhibitory effects than trastuzumab in dual ligand-stimulated spheroid growth. Enhanced inhibitory effects were observed when both drugs were combined (FIG. 4e).

In comparison to the double combination of MM-111 or trastuzumab with tamoxifen or fulvestrant, the triple combination of MM-111, trastuzumab and either tamoxifen (FIG. 4f) or fulvestrant (FIG. 4g) showed similar inhibitory effects to those of MM-111 and trastuzumab in combination (FIG. 4e) on estrogen- and heregulin-(dual ligand) stimulated spheroid growth.

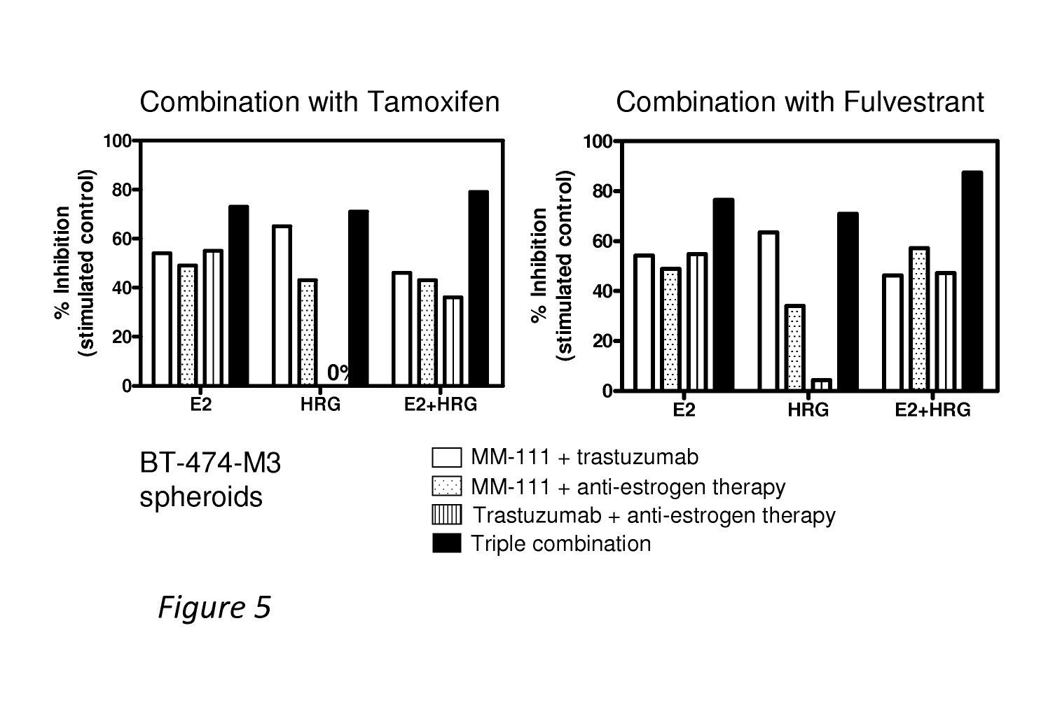

The data in the preceding Examples demonstrate that combination therapies comprising MM-111 and an anti-estrogen therapeutic are more effective than each of these therapies alone. The percent of spheroid growth inhibition induced by each treatment under estrogen or heregulin stimulation is summarized in FIG. 5 and Table 1. MM-11l was required for inhibition of spheroids stimulated with heregulin. For each stimulated condition tested, the triple combination resulted in the greatest inhibition of spheroid growth, providing a percent inhibition ranging from about 70% to about 90%.

TABLE 1
Percent inhibitor induced maximal spheroid growth inhibition

| | | | |
|---|---|---|---|
| MM-111 + | MM-111 + | Trastuzumab + | Triple |
| Trastuzumab | anti-estrogen | anti-estrogen | combination |

Tamoxifen combination

| | | | | |
|---|---|---|---|---|
| E2 | 54% | 49% | 55% | 73% |
| HRG | 65% | 43% | 0% | 71% |
| E2 + HRG | 46% | 43% | 36% | 79% |

Fulvestrant combination

| | | | | |
|---|---|---|---|---|
| E2 | 54% | 49% | 55% | 77% |
| HRG | 64% | 34% | 4% | 71% |
| E2 + HRG | 46% | 57% | 47% | 88% |

The percent of spheroid growth inhibition (normalized to untreated, stimulated control) was determined for 1 μM doses of inhibitor treatment.

The combination of MM-111 and tamoxifen resulted in potent inhibition of tumor growth in vivo. Taken together, these data demonstrate that the combination of MM-111 and anti-estrogen therapies results in potent anti-tumor effects in vitro and in vivo.

MM-111 in Combination with Lapatinib

### Methods

### Computational Modeling

A computational model of HRG-induced phospho-ErbB3 signaling, as well as a model of lapatinib, was used as previously described (Schoeberl, et al 2009).

### Cell Signaling Assay

Serum-starved cells are pre-incubated with serial dilutions of MM-111, lapatinib or combinations at doses and treatment times indicated, followed by stimulation with 5 nM heregulin 1-β (R&D Systems, Minneapolis, Minn.) for 10 minutes. Cell lysates are probed for phospho-ErbB3 (pErbB3), and phospho-AKT (pAKT) by ELISA as described previously (Schoeberl et al, 2009). Inhibitor IC₅₀ values are calculated by fitting dose-response data to a 4-parameter sigmoidal curve (GraphPad Prism®, GraphPad Software, Inc., La Jolla, Calif.).

### Cell Proliferation Assay

Cells (8,000/well) are seeded into 96-well plates and incubated overnight. Inhibitor is added at doses indicated and cells are treated for 24 hours. For experiments with ligand stimulation, cells are serum-starved overnight prior to addition of inhibitor and 2 nM heregulin 1-□ (R&D Systems, Minneapolis, Minn.) is added 1 hour post-inhibitor treatment in media containing 5% FBS. Numbers of viable cells are measured as an indicator of cell proliferation using the CellTiter-Glo® Luminescent Cell Viability Assay (Promega, Madison, Wis.).

### Apoptosis Assay

BT474-M3 cells (2000 cells/well) are plated in Ultra Low Cluster 96-well plate (Costar®, Corning, N.Y.). After overnight incubation, spheroids are treated with inhibitor at concentrations indicated for 72 hours. Spheroids are then trypsinized and combined with floating cells. Cells are washed twice with cold PBS and suspended in binding buffer (0.01 M HEPES, pH 7.4; 0.14 M NaCl; 2.5 mM CaCl₂). Cells are then stained with FITC-conjugated Annexin V and PI. Apoptotic cells are quantified on a FACSCalibur™ FACS machine.

### Xenograft Efficacy Studies

Tumor xenografts are established by subcutaneous injection of BT474-M3 cells into the flank of 5-6 weeks old female athymic nude mice (nu/nu; Charles River Labs, Wilmington, Mass.). Mice receive a subcutaneous 60 day, slow-release estrogen implant in the opposite flank (0.72 mg pellet; Innovation Research of America, Sarasota, Fla.) 24 hours prior to the injection of cells. Once tumors reach a mean volume of 150-500 mm³, mice are randomized into groups of 8 or 10 and dosed by intraperitoneal injection once every three days with vehicle, MM-111 or lapatinib. For lapatinib combination studies, MM-111 is given once every seven days and lapatinib daily by gavage at doses indicated.

### Aromatase-Overexpressing BT474-M3 Cells and Proliferation Assay

BT474-M3 cells were transfected with PS100010 vector containing human aromatase (gene accession No: NM_{—}000103.2). Cells with stable expression of aromatase (BT474-M3-Aro) were obtained after selection with 400 μg/ml geneticin. For cell proliferation assay, BT474-M3-Aro cells (5000 cells/well) were plated in phenol red-free RPMI-1640 medium containing 5% charcoal-stripped FBS into 96-well plate. After overnight incubation, indicated treatments were introduced in the presence of androstenedione (A-4; 200 nM) and heregulin (HRG; 2 nM). After three days of treatment, cell viability was determined by WST-1 (Roche; Cat. #11 644 807 001) according to manufacturer's instruction. Cell viability in the presence of 5% charcoal-stripped FBS was set as control (100%).

### Example 5

### The Combination of MM-111 and Lapatinib Inhibits Tumor Growth In Vivo

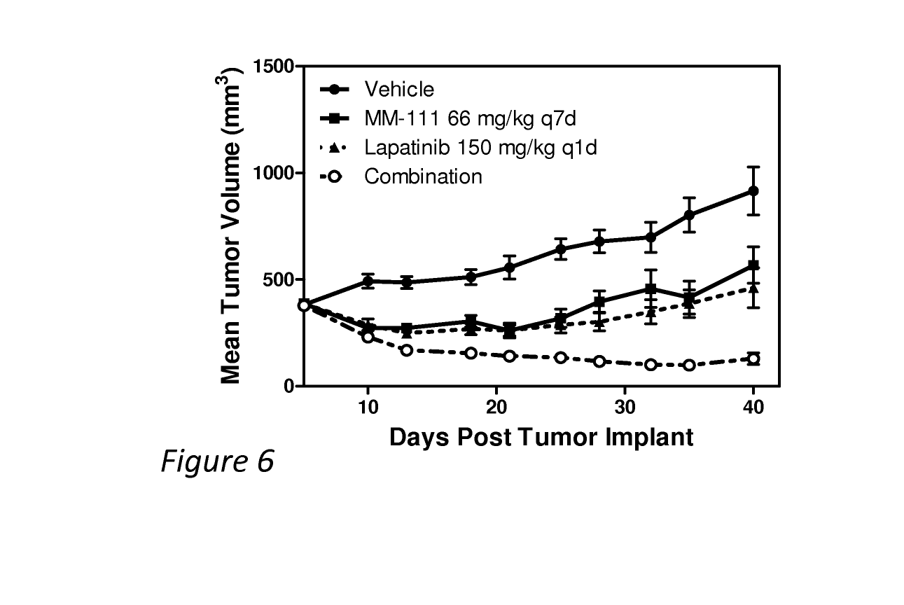

The combination of MM-111 with lapatinib was investigated in vivo in the BT474-M3 breast cancer xenograft model using the methods described above or minor variations thereof. MM-111 and lapatinib were each dosed at an optimal efficacious dose weekly and daily, respectively. The combination of MM-111 and lapatinib provided more potency compared to either drug alone, reaching statistical significance for MM-111 (p=3.9×10−4) and lapatinib (p=5.1×10−3) on day 13 (FIG. 6). The percent change in tumor volume from day 40 to day 7 (inoculation) was calculated for each group (FIG. 6b). The combination of MM-111 and lapatinib resulted in a percent change in tumor volume of −69% (about 70%), reflecting tumor regressions, compared to −11% (about 10%) for lapatinib and 14% (about 15%) for MM-111.

### Example 6

### Simulations Predict Lapatinib has Suboptimal Activity in Inhibiting Heregulin-Driven pErbB3 and pAKT

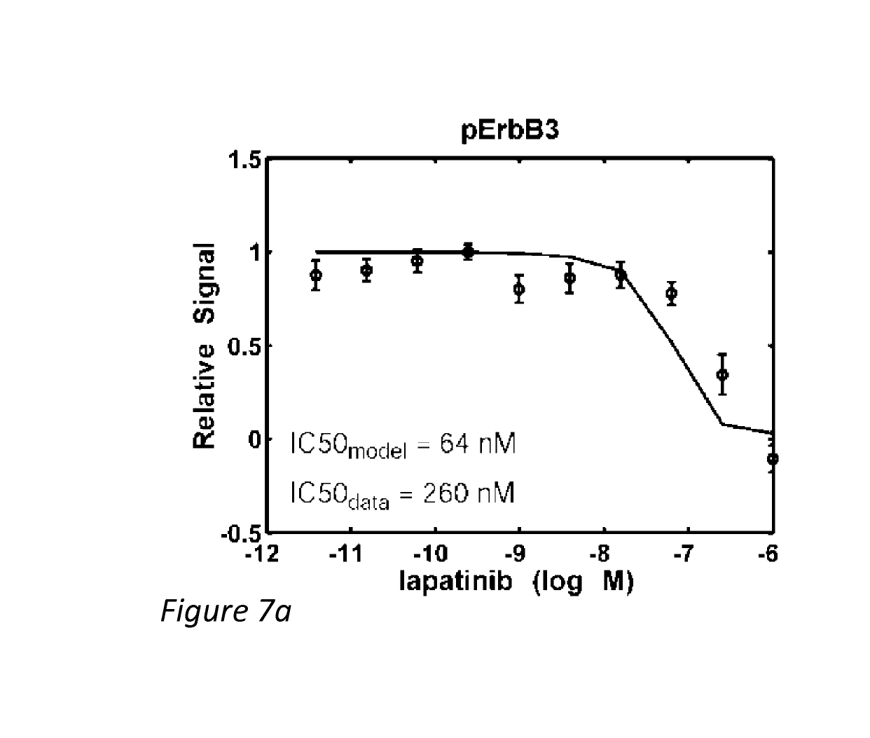

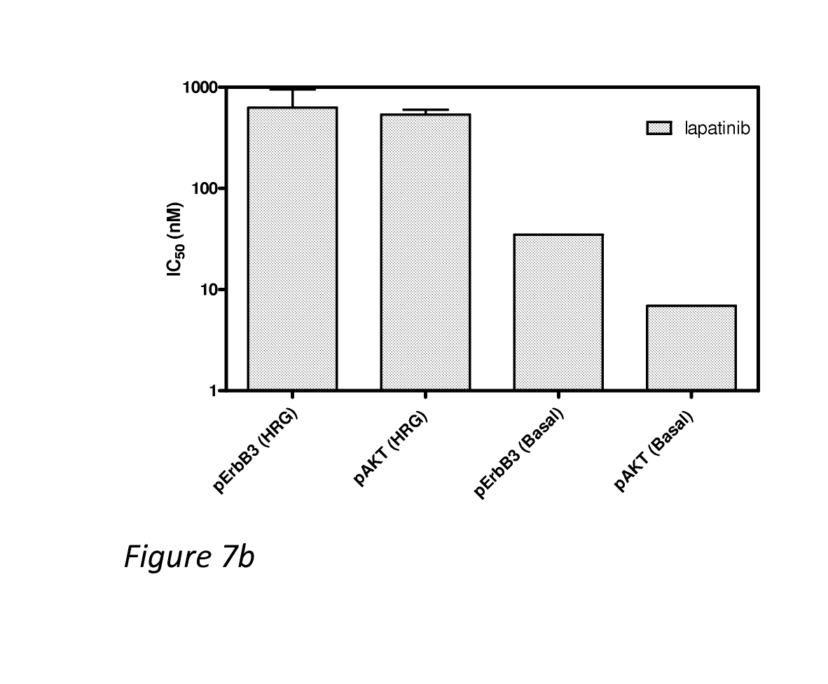

A dose range of lapatinib inhibition of pErbB3 activation was predicted using the computational modeling described above. A dose range of lapatinib was applied to BT474-M3 cells followed by stimulation with 5 nM heregulin for 10 min. The amount of pErbB3 was measured by ELISA using the methods described above or minor variations thereof. Model-generated dose-response curves overlay the experimental data (FIG. 7a). A comparison of the inhibitory activity of lapatinib in heregulin-stimulated or unstimulated (basal) cells was performed to demonstrate that heregulin signaling perturbs the activity of lapatinib. Untreated and heregulin-stimulated cells were probed for pErbB3 and pAKT and the IC50 was calculated (FIG. 7b). These data show that lapatinib alone is not an effective inhibitor of heregulin-activated signaling.

### Example 7

### MM-111 is a More Potent Inhibitor of HRG-Driven ErbB3 and AKT Phosphorylation than Lapatinib

In order to compare the ability of MM-111 and lapatinib to inhibit heregulin-induced ErbB3 activation, BT474-M3, or an additional ErbB2 overexpressing breast tumor cell line, ZR75-30 (ATCC® #

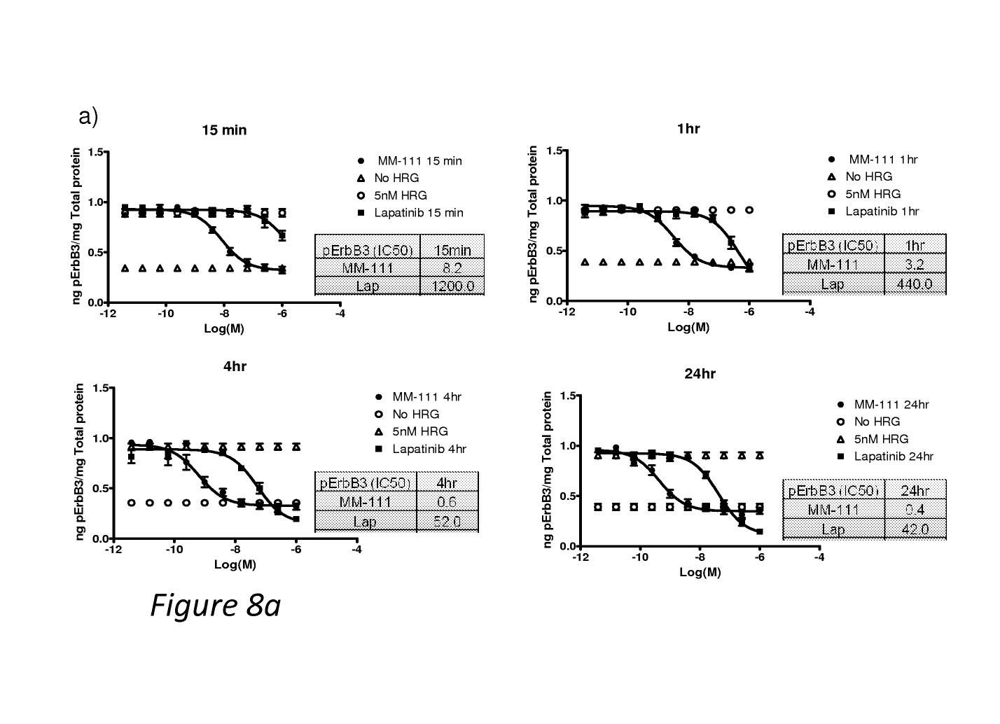

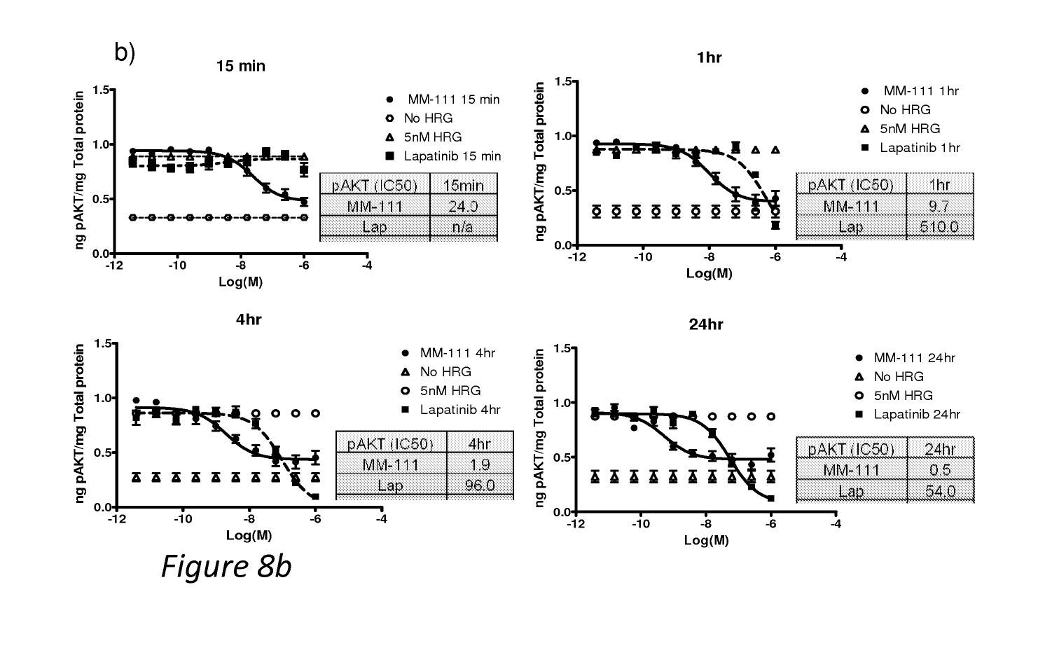

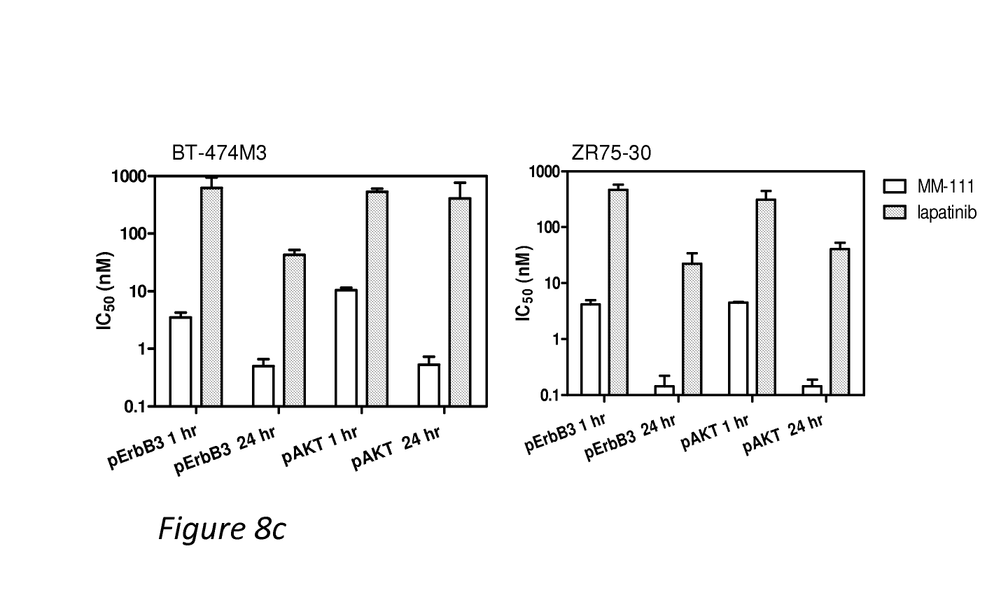

CRL-1504™), cells were incubated with serial dilutions of either inhibitor for 15 minutes, 1 hour, 4 hours, and 24 hours followed by stimulation with 5 nM heregulin for 10 min. Amounts of pAKT and pErbB3 were measured by ELISA essentially as described. MM-111 potently reduced pErbB3 levels (inhibited ErbB3 phosphorylation) in BT474-M3 (IC₅₀=3 nM) cells (FIG. 8a) and ZR75-30 cells (IC₅₀=5 nM) (FIG. 8c). Good reduction by MM-111 of pAKT levels (inhibition of AKT phosphorylation) in BT474-M3 (IC₅₀=10) (FIG. 8b) and in ZR75-30 cells (IC₅₀=4 nM) (FIG. 8d) was also observed. The ability of MM-111 to inhibit heregulin-induced ErbB3 activation (phosphorylation) was superior to lapatinib by greater than an order of magnitude and the relative IC₅₀ for each inhibitor (FIG. 8c) was consistent following up to 24 hours incubation with inhibitors, indicating treatment times had little effect on the potency of the inhibitors.

### Example 8

### The Combination of MM-111 and Lapatinib Potently Inhibits pAKT

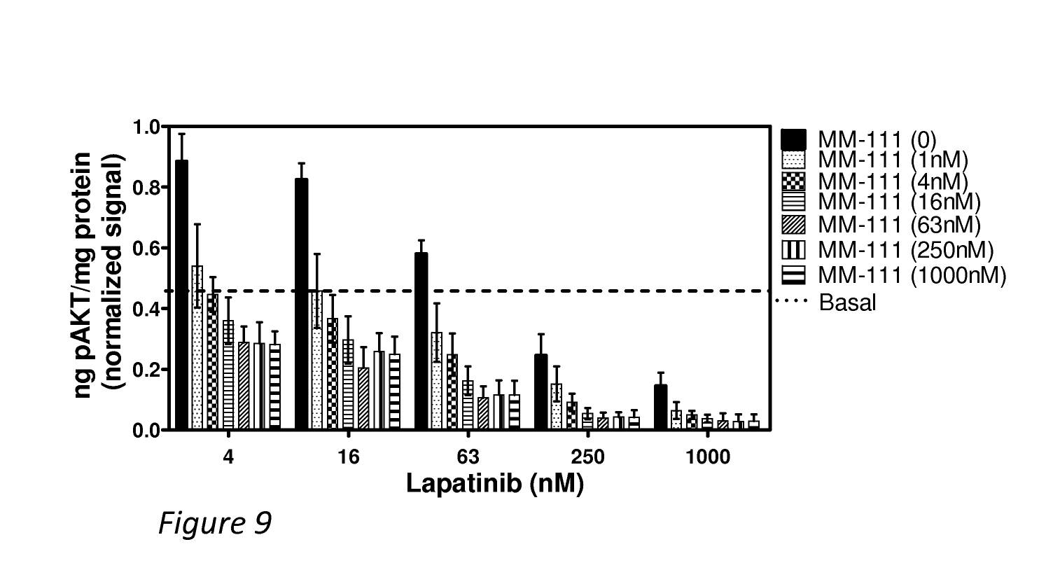

The effect of MM-111 combined with lapatinib on pAKT inhibition (reduction of pAKT levels) was assessed in heregulin-stimulated BT474-M3 cells. Cells were incubated for 2 hours with a dose range of MM-111, lapatinib or their combination and pAKT was measured by ELISA. In the presence of heregulin, the combination of MM-111 and lapatinib was extremely effective, inhibiting pAKT well below basal levels at therapeutically relevant concentrations (FIG. 9). Treatment with either MM-111 (1 μM) or lapatinib (1 μM) alone resulted in similar levels of pAKT inhibition (see FIG. 8b) while the combination resulted in about 20% more inhibition of pAKT.

### Example 9

### The Ability of Lapatinib to Inhibit Cell Proliferation is Perturbed Under Heregulin-Stimulated Conditions

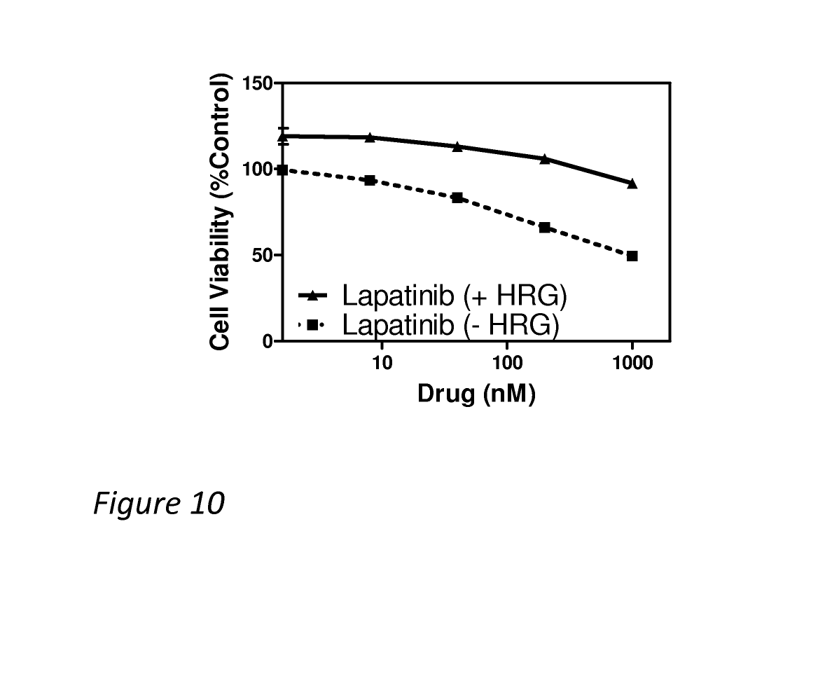

The effect of lapatinib on cell proliferation was measured in unstimulated and heregulin-stimulated BT474-M3 cells. Cells grown in serum or in serum plus 2 nM heregulin were treated with lapatinib across a dose range for 24 hours. Lapatinib treatment resulted in about a 50% inhibition of unstimulated cells but its effect was reduced to about 23% inhibition in heregulin-stimulated BT474-M3 cells (FIG. 10).

### Example 10

### Treatment with the Combination of MM-111 and Lapatinib Results in Increased Apoptosis

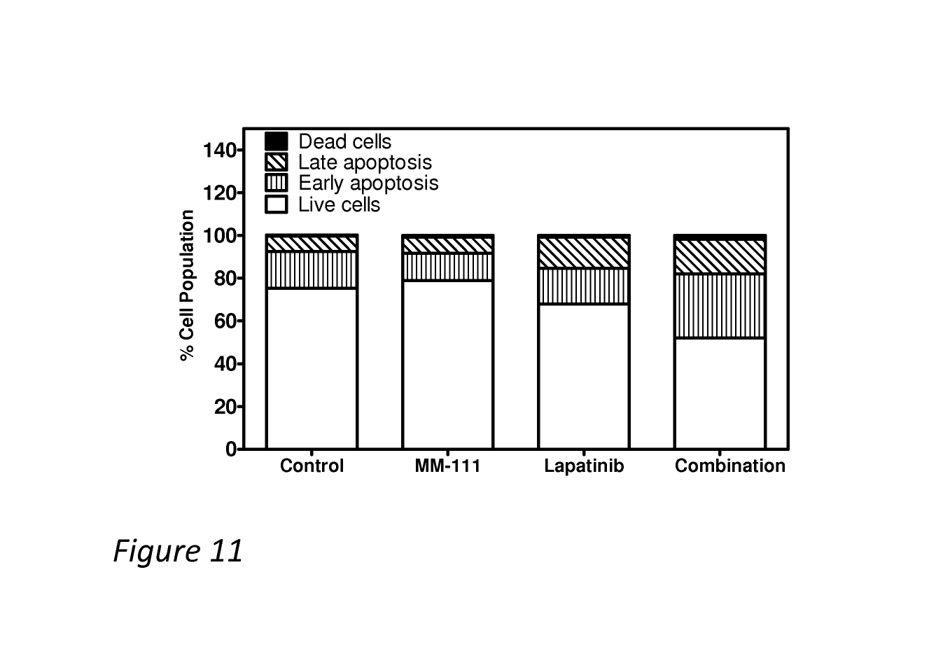

The effect of the MM-111 combination with lapatinib on apoptosis was assessed in a BT474-M3 spheroid model. Spheroids were prepared using the methods described above or minor variations thereof and treated with MM-111 (100 nM), lapatinib (33 nM), or a combination of 100 nM MM-111 and 33 nM lapatinib. Cells were then stained with Annexin V and propidium iodide (PI) and quantitated using FACS (FIG. 11, Table 2). Cell populations staining positive with Annexin V and PI were quantified as late apoptotic, cell populations staining positive with Annexin V but not PI were quantified as early apoptotic, cell populations staining positive for PI but not Annexin V were quantified as dead cells and populations of cells not stained with either Annexin V or PI were considered alive and not apoptotic (Table 2). Spheroids that were treated with both MM-111 and lapatinib had a higher number of total apoptotic cells (about 46%) compared to those treated with only lapatinib (about 31%) or only MM-111 (about 20%; FIG. 10).

TABLE 2
Percent cell population after treatment
with MM-111, lapatinib or the combination

| | Live cells | Early apoptosis | Late apoptosis | Dead cells |
|---|---|---|---|---|
| Control | 75.2 | 17.3 | 7.2 | 0.42 |
| MM-111 | 78.9 | 12.9 | 7.5 | 0.74 |
| Lapatinib | 67.9 | 16.8 | 14.5 | 0.73 |
| Combination | 52.1 | 30.0 | 16.2 | 1.74 |

### Example 11

### MM-111 Combines Positively with Anti-Estrogen Drugs and Lapatinib in Inhibiting Dual Ligand (Estrogen and Heregulin)-Stimulated Spheroid Growth

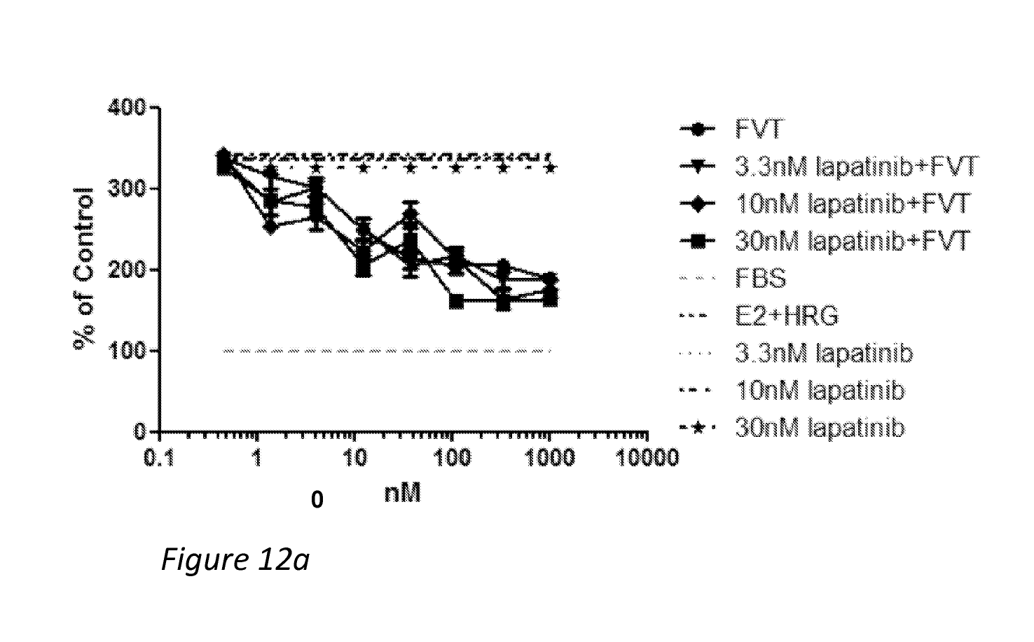

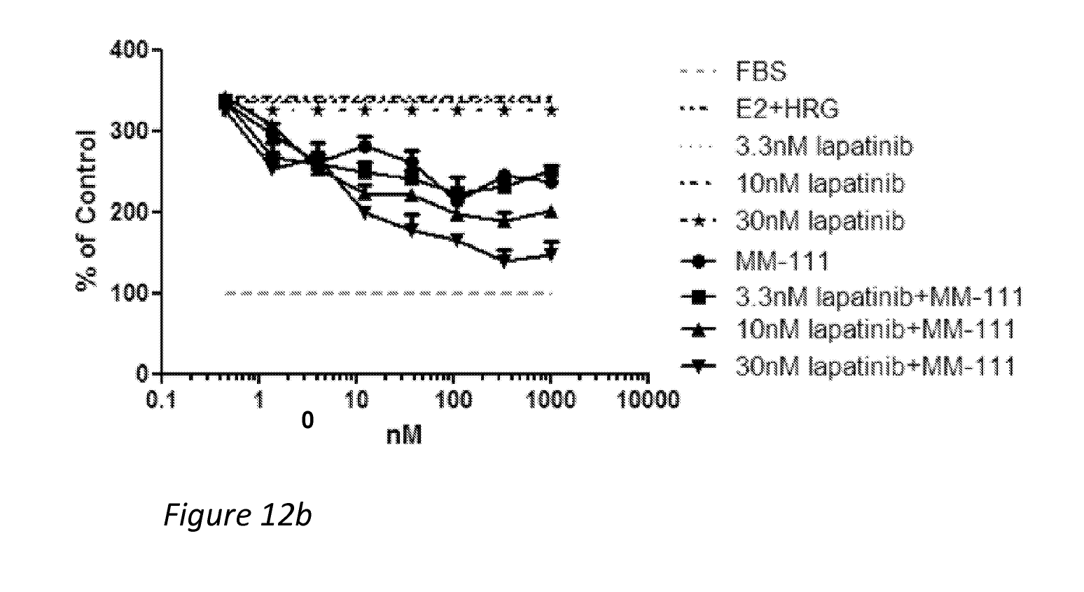

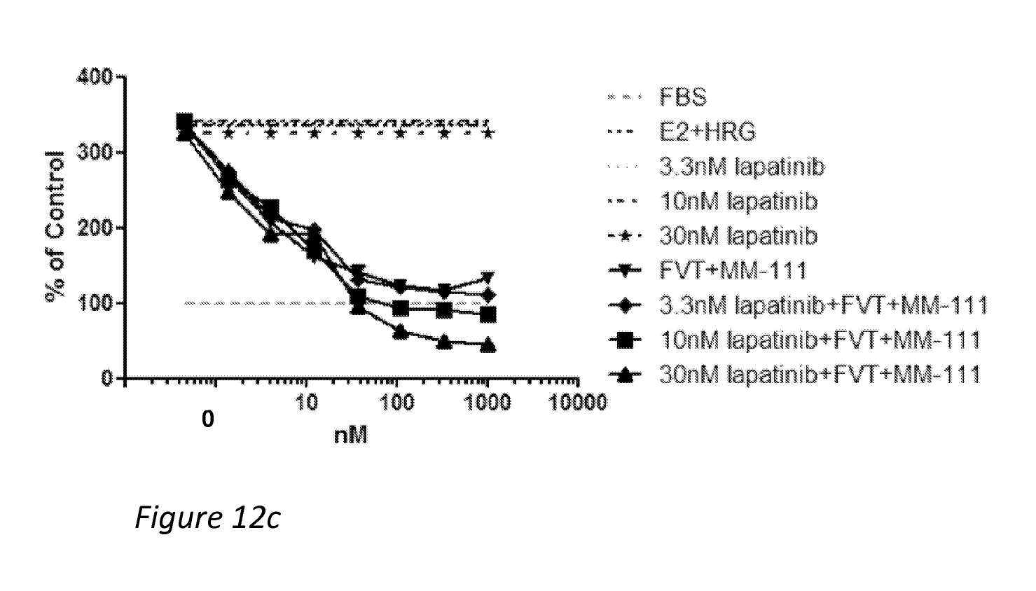

To further investigate the ability of MM-111 to inhibit cell growth when in combination with both anti-estrogen drugs and tyrosine kinase inhibitors, spheroids of estrogen and heregulin-stimulated BT474-M3 cells were prepared using the methods described above or minor variations thereof and treated with 3.3 nM, 10 nM, or 30 nM lapatinib, either alone or in combination with a dose range of fulvestrant (FVT) (FIG. 12a); 3.3 nM, 10 nM, or 30 nM lapatinib, either alone or in combination with a dose range of MM-111 (FIG. 12b); or 3.3 nM, 10 nM, or 30 nM lapatinib, either alone or in combination with a dose range of both MM-111 and fulvestrant (FIG. 12c). In the presence of dual ligand stimulation the combination of lapatinib and FVT did not greatly increase inhibition of spheroid growth over lapatinib alone (FIG. 12a). In contrast, the addition of MM-111 greatly increased the sensitivity of the spheroids to lapatinib treatment (FIG. 12b), and the triple combination of lapatinib, FVT and MM-111 showed an even greater increase of spheroid growth inhibition over lapatinib alone.

### Example 12

### MM-111 Combines Positively with Anti-Estrogen Drugs in Inhibiting Spheroid Growth in BT474-M3 Cells Overexpressing Human Androstenedione

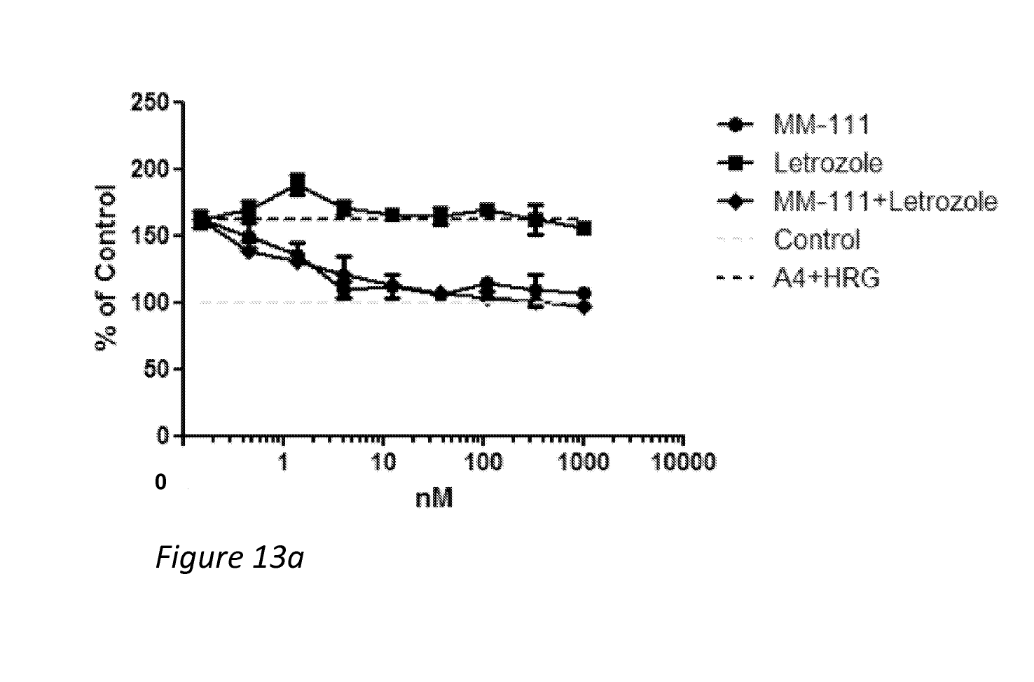

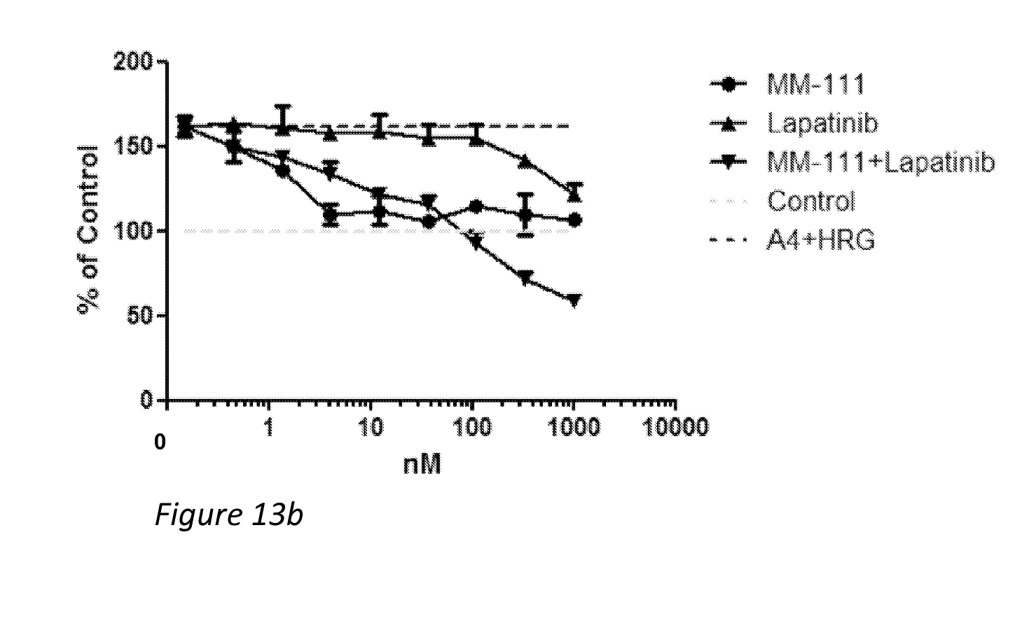

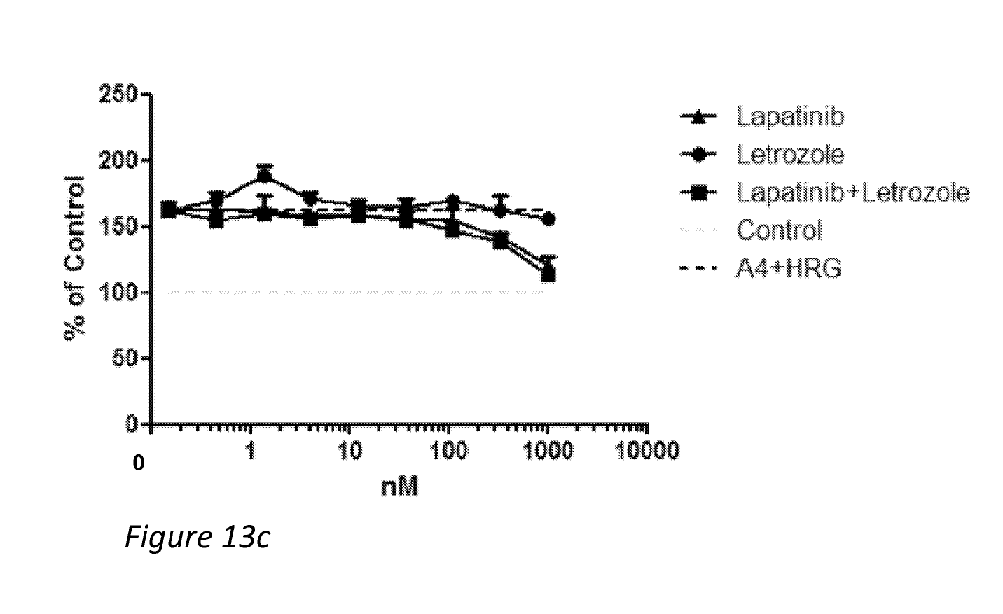

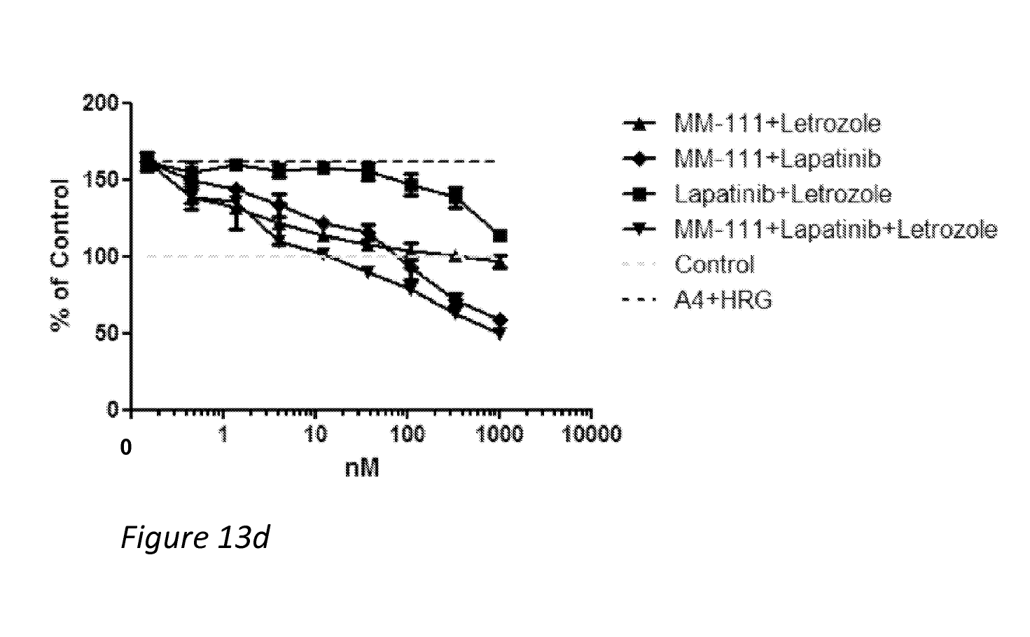

Androstenedione is a steroid hormone that is converted to estrogen by aromatase. To further investigate the ability of MM-111 to inhibit spheroid growth, aromatase-expressing cells were treated in the presence of androstenedione (A4) and heregulin (HRG) with MM-111, letrozole, or the combination of MM-111 or letrozole (FIG. 13a); MM-111, lapatinib, or the combination of MM-111 and lapatinib (FIG. 13b); lapatinib, letrozole, or the combination of lapatinib and letrozole (FIG. 13c); and each of the dual combination plus the triple combination of MM-111, lapatinib, and letrozole (FIG. 13d). In cells treated with A4 and HRG, the letrozole treatment did not result in significant inhibition of spheroid cell growth as compared to control (untreated) cells, whereas cells treated with MM-111 alone or the combination of MM-111 and letrozole inhibited cell proliferation to a similar extent (FIG. 13a). Lapatinib treatment of the cells did not result in growth inhibition except at high concentrations, whereas treatment with MM-111 alone or in combination resulted in similar levels of cell growth inhibition except in higher concentrations where the combination showed increased inhibition of cell growth over either of the single treatments (FIG. 13b). Treatment with lapatinib alone, letrozole alone, or the combination of lapatinib and letrozole did not result in significant cell growth inhibition except at high concentration (FIG. 13c). Similarly, as shown in FIG. 13d, the double combination of lapatinib and letrozole resulted in cell growth inhibition only at high drug concentration. In contrast the dual combinations of MM-111 and letrozole or MM-111 and lapatinib both showed an increase in cell growth inhibition as compared to control, and the triple combination of MM-111, lapatinib, and letrozole inhibited cell growth to an even greater degree.

### Example 13

### Amino Acid Sequence of MM-111(SEQ ID NO:1)

QVQLQESGGGLVKPGGSLRLSCAASGFTFSSYWMSWVRQAPGKGLEWVANINRDGSASYYVD
SVKGRFTISRDDAKNSLYLQMNSLRAEDTAVYYCARDRGVGYFDLWGRGTLVTVSSASTGGGG
SGGGGSGGGGSQSALTQPASVSGSPGQSITISCTGTSSDVGGYNFVSWYQQHPGKAPKLMIYDVS
DRPSGVSDRFSGSKSGNTASLIISGLQADDEADYYCSSYGSSSTHVIFGGGTKVTVLGAASDAHK
SEVAHRFKDLGEENFKALVLIAFAQYLQQSPFEDHVKLVNEVTEFAKTCVADESAENCDKSLHT
LFGDKLCTVATLRETYGEMADCCAKQEPERNECFLQHKDDNPNLPRLVRPEVDVMCTAFHDNE
ETFLKKYLYEIARRHPYFYAPELLFFAKRYKAAFTECCQAADKAACLLPKLDELRDEGKASSAK
QRLKCASLQKFGERAFKAWAVARLSQRFPKAEFAEVSKLVTDLTKVHTECCHGDLLECADDRA
DLAKYICENQDSISSKLKECCEKPLLEKSHCIAEVENDEMPADLPSLAADFVESKDVCKNYAEAK
DVFLGMFLYEYARRHPDYSVVLLLRLAKTYETTLEKCCAAADPHECYAKVFDEFKPLVEEPQNL
IKQNCELFEQLGEYKFQNALLVRYTKKVPQVSTPTLVEVSRNLGKVGSKCCKHPEAKRMPCAED
YLSVVLNQLCVLHEKTPVSDRVTKCCTESLVNRRPCFSALEVDETYVPKEFQAETFTFHADICTL
SEKERQIKKQTALVELVKHKPKATKEQLKAVMDDFAAFVEKCCKADDKETCFAEEGKKLVAAS
QAALGLAAALQVQLVQSGAEVKKPGESLKISCKGSGYSFTSYWIAWVRQMPGKGLEYMGLIYP
GDSDTKYSPSFQGQVTISVDKSVSTAYLQWSSLKPSDSAVYFCARHDVGYCTDRTCAKWPEWL
GVWGQGTLVTVSSGGGGSSGGGSGGGGSQSVLTQPPSVSAAPGQKVTISCSGSSSNIGNNYVSW
YQQLPGTAPKLLIYDHTNRPAGVPDRFSGSKSGTSASLAISGFRSEDEADYYCASWDYTLSGWV
FGGGTKLTVLG

Dosing and administration of MM-111 in combination one or more additional therapeutics

### Example 14

### Mode of Administration of MM-111

MM-111 is prepared as a formulation containing 25 mg/ml MM-111 in a sterile aqueous solution comprising 20 mM L-histidine hydrochloride, 150 mM sodium chloride, pH 6.5, which is stored at 2-8° C.

MM-111 must be brought to room temperature prior to administration. Containers (e.g., vials) of MM-111 must not be shaken. The appropriate quantity of MM-111 is removed from the container, diluted in 250 mL of 0.9% normal saline and administered as an infusion using a low protein binding in-line filter (e.g., a 0.22 micrometer filter).

MM-111 is initially administered over about 90 minutes (first administration). In the absence of an infusion reaction, subsequent doses are administered over about 60 minutes.

A patient's body weight at the start of a dosing cycle is used to calculate the dose used throughout the cycle. Should a patient's body weight change by more than 10%, a new total dose is calculated to reflect this change.

### Example 15

### Dosage and Administration of MM-111

Preferred plasma concentrations of MM-111 achieved during treatment are at least 106 mg/L. It has now been discovered that certain combinations of dose frequency and dosage will achieve and maintain this plasma concentration during the course of treatment in at least half, and preferably in more than 60%, 70% or 80% of treated patients.

In certain embodiments a higher initial dose (loading dose—LD) is given, followed as defined intervals by at least one maintenance dose (MD). Intervals of dosing in days are typically indicated as QxD, wherein x represents an integer, so that a QxD of 7 indicates dosing every 7 days. Table 3A, Table 3B, and Table 3C below show doses and dosing intervals of the invention. In Table 3A, Table 3B, and Table 3C the indicated loading doses are optional—initial doses are preferably made at the indicated loading dose (LD), but may (e.g., as directed or at the physician's discretion) be made at the maintenance dose (MD). Table 3A provides a set of exemplary dosing intervals, loading doses and maintenance doses. Table 3B provides a variation of Table 3A allowing for dosage variability (indicated as “about”) of up to +/−3 mg/mL. Table 3C appears below and provides a more extensive set of exemplary dosing intervals, loading doses and maintenance doses. In each cell of Table 3A, Table 3B, and Table 3C, the top figure is the integer x in the interval QxD (e.g., 18 as the top figure in a cell indicates a dosing interval of Q18D or every 18 days), the middle figure represents the (optional) loading dose (LD) in mg/kg, nd the bottom figure represents the maintenance dose (MD) in mg/kg. Thus the top cell in Table 3A indicates a dosing interval (QxD) of once every seven days, a loading dose (optional) of 25 mg per kg of patient body weight, and a maintenance dose of 20 mg per kg of patient body weight; while the cell furthest to the right on the top row of Table 3C indicates a dosing interval (QxD) of once every seven days, a loading dose (optional) of 30 mg per kg of patient body weight, and a maintenance dose of 15 mg per kg of patient body weight.

TABLE 3A
7
25
20
7
40
30
14
60
45
14
90
75
21
120
105

```
TABLE 3B
  7
about 25
about 20
  7
about 40
about 30
14
about 60
about 44
14
about 90
about 75
21
  about 120
  about 105
```

TABLE 3C

| | | | | | | | | | | | | |
|---|---|---|---|---|---|---|---|---|---|---|---|---|
| 7 | 7 | 7 | 7 | 7 | 7 | 7 | 7 | 7 | 7 | 7 | 7 | 7 |
| 10 | 15 | 20 | 25 | 30 | 15 | 20 | 25 | 30 | 35 | 20 | 25 | 30 |
| 5 | 5 | 5 | 5 | 5 | 10 | 10 | 10 | 10 | 10 | 15 | 15 | 15 |
| 7 | 7 | 7 | 7 | 7 | 7 | 7 | 7 | 7 | 7 | 7 | 7 | 7 |
| 35 | 40 | 25 | 30 | 35 | 40 | 45 | 30 | 35 | 40 | 45 | 50 | 55 |
| 15 | 15 | 20 | 20 | 20 | 20 | 20 | 25 | 25 | 25 | 25 | 25 | 25 |
| 7 | 7 | 14 | 14 | 14 | 14 | 14 | 14 | 14 | 14 | 14 | 14 | 14 |
| 60 | 65 | 35 | 40 | 45 | 50 | 55 | 60 | 65 | 70 | 75 | 40 | 45 |
| 25 | 25 | 30 | 30 | 30 | 30 | 30 | 30 | 30 | 30 | 30 | 35 | 35 |
| 14 | 14 | 14 | 14 | 14 | 14 | 14 | 14 | 14 | 14 | 14 | 14 | 14 |
| 50 | 55 | 60 | 65 | 70 | 75 | 45 | 50 | 55 | 60 | 65 | 70 | 75 |
| 35 | 35 | 35 | 35 | 35 | 35 | 40 | 40 | 40 | 40 | 40 | 40 | 40 |
| 14 | 14 | 14 | 14 | 14 | 14 | 14 | 14 | 14 | 14 | 14 | 14 | 14 |
| 50 | 55 | 60 | 65 | 70 | 75 | 55 | 60 | 65 | 70 | 75 | 60 | 65 |
| 45 | 45 | 45 | 45 | 45 | 45 | 50 | 50 | 50 | 50 | 50 | 55 | 55 |
| 14 | 14 | 14 | 14 | 14 | 14 | 14 | 14 | 21 | 21 | 21 | 21 | 21 |
| 70 | 75 | 65 | 70 | 75 | 70 | 75 | 75 | 60 | 65 | 70 | 65 | 70 |
| 55 | 55 | 60 | 60 | 60 | 65 | 65 | 70 | 55 | 55 | 55 | 60 | 60 |
| 21 | 21 | 21 | 21 | 21 | 21 | | | | | | | |
| 75 | 70 | 75 | 80 | 85 | 90 | | | | | | | |
| 60 | 65 | 70 | 75 | 80 | 85 | | | | | | | |

### Example 16

### Dosage and Administration of MM-111 with Lapatinib and Trastuzumab

Treatment for patients with trastuzumab-refractory HER2-overexpressing breast cancer is a critical unmet need in the field of breast oncology, and novel approaches to address this need are required. Although selective tyrosine kinase inhibitors (TKIs) have been highly effective for the treatment of certain tyrosine kinase oncogene-driven cancers, their clinical anti-tumor efficacy in the treatment of HER2-driven breast cancer has been disappointing despite adequate biodistribution and apparent target inhibition. Two completed phase II trials using the most potent HER2 TKI, lapatinib, have reported response rates of only 4%-8% in patients with trastuzumab-refractory HER2-overexpressing breast cancer. It is now known that the effective treatment of HER2+ breast cancer is more complex and resilient than previously thought. Recent evidence has highlighted the role of HER3 and a robust signal buffering capacity inherent in the HER2-HER3 tumor driver that protects it against a two log inhibition of HER2 catalytic activity, placing it beyond the therapeutic index of even the most potent tyrosine kinase inhibitors (TKIs).

Typically, lapatinib is administered at a dosage of 1000 to 1500 mg in 250 mg tablets taken once daily. Lapatinib is often used in combination with another cancer medication, capecitabine, which is taken for 14 day periods with one week in between.

In order to test whether the full inactivation of the HER2-HER3 driver can be achieved with much higher TKI dosing at an intermittent dosing schedule is more efficacious than continuous dosing, a modified dosing schedule is used wherein an increased dose of lapatinib is administered on days 1-5 of a 14 day cycle, said increased dose being a higher dose than the standard dose of 1000 to 1500 mg/day. In some embodiments, the higher lapatinib dose is between 2000 and 9000 mg/d. For example, higher lapatinib dose might be 2000, 2250, 3375, 3000, 3250, 3500, 3750, 4000, 4250, 4500, 4750, 5000, 5250, 5500, 5750, 6000, 6250, 6500, 6750, 7000, 7250, 7500, 7750, 8000, 8250, 8500, 8750, or 9000 mg/day, and so on.

In certain embodiments a loading dose is given on day 1 of the 14-day cycle that is a higher dose than that given on subsequent days, the maintenance dose. For example, a loading dose given on day 1 of the 14 day cycle might be 7000 mg/day, followed by a maintenance dose of 3000 mg/day. Non-limiting examples of loading dose and maintenance dose combinations are listed in Table 4 below.

MM-111 is administered as described in Example 15. In some embodiments the treatment further comprises trastuzumab. Trastuzumab is typically given with an initial loading dose followed by a maintenance dose. For example, trastuzumab may be dosed at a loading dose of 8 mg/kg followed by a maintenance dose of 6 mg/kg every three weeks.

TABLE 4
Exemplary lapatinib dosing schedule: loading dose (top
number) and maintenance dose (bottom number) in mg/d

| | | | | | | | | | | | | |
|---|---|---|---|---|---|---|---|---|---|---|---|---|
| 2000 | 2000 | 2000 | 2500 | 2500 | 2500 | 3000 | 3000 | 3000 | 3000 | 3000 | 3500 | 3500 |
| 1000 | 1500 | 2000 | 1000 | 1500 | 2000 | 1000 | 1500 | 2000 | 2500 | 3000 | 1000 | 1500 |
| 3500 | 3500 | 3500 | 4000 | 4000 | 4000 | 4000 | 4000 | 4000 | 4500 | 4500 | 4500 | 4500 |
| 2000 | 2500 | 3000 | 1000 | 1500 | 2000 | 2500 | 3000 | 3500 | 1000 | 1500 | 2000 | 2500 |
| 4500 | 4500 | 4500 | 5000 | 5000 | 5000 | 5000 | 5000 | 5000 | 5000 | 5000 | 5500 | 5500 |
| 3000 | 3500 | 4000 | 1000 | 1500 | 2000 | 2500 | 3000 | 3500 | 4000 | 4500 | 1000 | 1500 |
| 5500 | 5500 | 5500 | 5500 | 5500 | 5500 | 5500 | 6000 | 6000 | 6000 | 6000 | 6000 | 6000 |
| 2000 | 2500 | 3000 | 3500 | 4000 | 4500 | 5000 | 1000 | 1500 | 2000 | 2500 | 3000 | 3500 |
| 6000 | 6000 | 6000 | 6000 | 7500 | 7500 | 7500 | 7500 | 7500 | 7500 | 7500 | 7500 | 7500 |
| 4000 | 4500 | 5000 | 5500 | 1000 | 1500 | 2000 | 2500 | 3000 | 3500 | 4000 | 4500 | 5000 |
| 7500 | 7500 | 7500 | 7500 | 8000 | 8000 | 8000 | 8000 | 8000 | 8000 | 8000 | 8000 | 8000 |
| 5500 | 6000 | 6500 | 7000 | 1000 | 1500 | 2000 | 2500 | 3000 | 3500 | 4000 | 4500 | 5000 |
| 8000 | 8000 | 8000 | 8000 | 8000 | 9000 | 9000 | 9000 | 9000 | 9000 | 9000 | 9000 | 9000 |
| 5500 | 6000 | 6500 | 7000 | 7500 | 1000 | 1500 | 2000 | 2500 | 3000 | 3500 | 4000 | 4500 |
| 9000 | 9000 | 9000 | 9000 | 9000 | 9000 | 9000 | 9000 | | | | | |
| 5000 | 5500 | 6000 | 6500 | 7000 | 7500 | 8000 | 8500 | | | | | |

### Example 17

### Dosage and Administration of MM-111 with Cisplatin, Capecitabine, and Trastuzumab

Administration of MM-111 with cisplatin, capecitabine, and trastuzumab is done, for example, by the following method or minor variations thereof.

Patients are administered therapy on a 21-day treatment cycle. Cisplatin is administered on day 1 of each 21-day cycle by intravenous (i.v.) infusion over two hours, at a dose of 80 mg/m². Capecitabine is administered orally, twice daily, at a dose of 1000 mg/m². Up to 21-day cycles of cisplatin and capecitabine are administered. Trastuzumab is administered i.v. at week 1 at an 8 mg/kg loading dose over 90 minutes, followed by a maintenance dose of 6 mg/kg every 21 days over 30-90 minutes. MM-111 is administered as described in the above Examples. For example, MM-111 is administered i.v. over 90 minutes for the first dose and then weekly over 60 minutes thereafter.

### Example 18

### Dosage and Administration of MM-111 with Lapatinib and Trastuzumb

Administration of MM-111 with lapatinib and trastuzumab is done, for example, by the following method or minor variations thereof. Trastuzumab is administered i.v. at a 4 mg/kg loading dose on week 1 over 90 minutes, followed by a 2 mg/kg weekly maintenance dose thereafter. Lapatinib is given by mouth either at 1000 mg daily doses or at the one of the dose regimens described in Example 13. MM-111 is administered as described in the above Examples. For example, MM-111 is administered i.v. over 90 minutes for the first dose and then weekly over 60 minutes thereafter.

### Example 19

### Dosage and Administration of MM-111 with Paclitaxel and Trastuzumab

Administration of MM-111 with paclitaxel and trastuzumab is done, for example, by the following method or minor variations thereof. Patients are administered therapy on a 28-day treatment cycle. Paclitaxel dosing begins on day 1 of cycle 1. Paclitaxel is administered at 80 mg/m² weekly, as an i.v. infusion over 60 minutes. Trastuzumab is administered at a 4 mg/kg loading dose on week 1, i.v. over 90 minutes, followed by a 2 mg/kg weekly maintenance dose thereafter. MM-111 is administered as described in the above Examples. For example, MM-111 is administered i.v. over 90 minutes for the first dose and then weekly over 60 minutes thereafter.

### Endnotes

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth.

All patents patent applications and publications mentioned herein are incorporated by reference to the same extent as if each independent patent or patent application was specifically and individually indicated to be incorporated by reference in its entirety.

### APPENDIX

### Anticancer Agents

A bispecific anti-ErbB2/anti-ErbB3 antibody co-administered in combination with an anti-estrogen receptor agent or a receptor tyrosine kinase inhibitor can be further co-administered with at least a third antineoplastic agent selected from any of those disclosed below.

TABLE 5
Exemplary antineoplastic agents for treatment of breast cancer in
combination with a bispecific anti-ErbB2/anti-ErbB3 antibody.

| | Exemplary Agent | | |
|---|---|---|---|
| Therapeutic Class | (Generic/Tradename) | Exemplary Dose | |
| Mitotic Inhibitors | paclitaxel (TAXOL ®; | 175 | mg/m² |
| | ABRAXANE ®) | | |
| | docetaxel | 60-100 | mg/m² |
| | (TAXOTERE ®) | | |
| Topoisomerase Inhibitors | camptothecin | | |
| | topotecan hydrochloride | | |
| | (HYCAMTIN ®) | | |
| | etoposide (EPOSIN ®) | | |
| Alkylating Agents | cyclophosphamide | 600 | mg/m² |
| | (CYTOXAN ®) | | |
| Platinum-Based Agents | Cisplatin | 20-100 | mg/m² |
| | carboplatin | 300 | mg/m² |
| | (PARAPLATIN ®) | | |
| | nedaplatin (AQUPLA ®) | | |
| | oxaliplatin | 65-85 | mg/m² |
| | (ELOXATIN ®) | | |
| | satraplatin (SPERA ®) | | |
| | triplatin tetranitrate | | |
| Selective Estrogen | tamoxifen | 20-40 | mg/day |
| Modulators (SERM) | (NOLVADEX ®) | | |
| | raloxifene (EVISTA ®) | 60 | mg/day |
| | toremifene | | |
| | (FARESTON ®) | | |
| Antimetabolites | methotrexate | 40 | mg/m² |
| | Fluorouracil (5-FU) | 500 | mg/m² |
| | Raltitrexed | | |
| Antitumor Antibiotics | Doxorubicin | 40-75 | mg/m² |
| | (ADRIAMYCIN ®) | | |
| | epirubicin | 60-120 | mg/m² |
| | (ELLENCE ®) | | |
| Aromatase Inhibitors | aminoglutethimide | 250-2000 | mg/day |
| | (CYTADREN ®) | | |
| | anastrozole | 1 | mg/day |
| | (ARIMIDEX ®) | | |
| | letrozole (FEMARA ®) | 2.5 | mg/day |
| | Vorozole | | |
| | exemestane | 25-50 | mg/day |
| | (AROMASIN ®) | | |
| | Testolactone | | |
| | fadrozole (AFEMA ®) | | |
| Anti-VEGF Agents | bevacizumab | 10 | mg/kg |
| | (AVASTIN ®) | | |
| Anti-ErbB2 (HER2/neu) | trastuzumab | 2-8 | mg/kg |
| Agents | (HERCEPTIN ®) | | |
| | Pertuzumab | | |
| | (OMNITARG ®) | | |
| Anti-ErbB3 (HER3) | U3-1287 (AMG 888) | | |
| Agents | | | |

APPENDIX
ANTICANCER AGENTS

| Other anticancer agents for combination with | | |
|---|---|---|
| a bispecific anti-ErbB2/anti-ErbB3 antibody | Brand Name(s) | Manufacturer/Proprietor |
| Anti-IGFIR Antibodies | | |
| AMG 479 (fully humanized mAb) | | Amgen |
| IMCA12 (fully humanized mAb) | | ImClone |
| NSC-742460 | | Dyax |
| 19D12 (fully humanized mAb) | | |
| CP751-871 (fully humanized mAb) | | Pfizer |
| H7C10 (humanized mAb) | | |
| alphaIR3 (mouse) | | |
| scFV/FC (mouse/human chimera) | | |
| EM/164 (mouse) | | |
| MK-0646, F50035 | | Pierre Fabre Medicament, Merck |
| Small Molecules Targeting IGF1R | | |
| NVP-AEW541 | | Novartis |
| BMS-536,924 (1H-benzoimidazol-2-yl)- | | Bristol-Myers Squibb |
| 1H-pyridin-2-one) | | |
| BMS-554,417 | | Bristol-Myers Squibb |
| Cycloligan | | |
| TAE226 | | |
| PQ401 | | |
| Anti-EGFR Monoclonal Antibodies | | |
| INCB7839 | | Incyte |
| Bevacizumab | Avastin ® | Genentech |
| Cetuximab | Erbitux ® | IMCLONE |
| mAb 806 | | |
| Matuzumab (EMD72000) | | |
| Nimotuzumab (TheraCIM) | | |
| Panitumumab | Vectibix ® | Amgen |
| Anti-ErbB3 Therapeutics | | |
| U3-1287/AMG888 | | U3 Pharma/Amgen |
| MM-121 | | Merrimack Pharmaceuticals |
| Anti-ErbB2 Therapeutics | | |
| trastuzumab | Herceptin ® | Genentech |
| HKI-272 - neratinib | | Wyeth |
| KOS-953 - tanespimycin | | Kosan Biosciences |
| Her/ErbB Dimerization Inhibitors | | |
| 2C4, R1273 - Pertuzumab | Omnitarg ® | Genentech, Roche |
| Small Molecules Targeting EGFR | | |
| CI-1033 (PD 183805) | | Pfizer, Inc. |
| EKB-569 | | |
| Gefitinib | IRESSA ™ | AstraZeneca |
| Lapatinib (GW572016) | | GlaxoSmithKline |
| Lapatinib Ditosylate | Tykerb ® | SmithKline Beecham |
| Erlotinib HCl (OSI-774) | Tarceva ® | OSI Pharms |
| PD158780 | | |
| PKI-166 | | Novartis |
| Tyrphostin AG 1478 (4-(3-Chloroanillino)- | | |
| 6,7-dimethoxyquinazoline) | | |
| Anti-cmet Antibody Therapies | | |
| AVEO (AV299) | | AVEO |
| AMG102 | | Amgen |
| 5D5 (OA-5D5) | | Genentech |
| Small Molecules Targeting cmet | | |
| PHA665752 | | |
| ARQ-650RP | | ArQule |
| ARQ 197 | | ArQule |
| Alkylating Agents | | |
| BCNU→ 1,3-bis t2-chloroethyl)- | | |
| nitrosourea | | |
| Bendamustine | | |
| Busulfan | Myleran | GlaxoSmithKline |
| Carboplatin | Paraplatin | Bristol-Myers Squibb |
| Carboquone | | |
| Carmustine | | |
| CCNU→ 1,-(2-chloroethyl)-3-cyclohexyl- | | |
| 1-nitrosourea (methyl CCNU) | | |
| Chlorambucil | Leukeran ® | Smithkline Beecham |
| Chlormethine | | |
| Cisplatin (Cisplatinum, CDDP) | Platinol | Bristol-Myers |
| Cyclophosphamide | Cytoxan | Bristol-Myers Squibb |
| Dacarbazine (DTIC) | Neosar | Teva Parenteral |
| Fotemustine | | |
| Hexamethylmelamine (Altretamine, HMM) | Hexalen ® | MGI Pharma, Inc. |
| Ifosfamide | Mitoxana ® | ASTA Medica |
| Lomustine | | |
| Mannosulfan | | |
| Melphalan | Alkeran ® | GlaxoSmithKline |
| Nedaplatin | | |
| Nimustine | | |
| Oxaliplatin | Eloxatin ® | Sanofi-Aventis US |
| Prednimustine, | | |
| Procarbazine HCL | Matulane | Sigma-Tau Pharmaceuticals, Inc. |
| Ribonucleotide Reductase Inhibitor (RNR) | | |
| Ranimustine | | |
| Satraplatin | | |
| Semustine | | |
| Streptozocin | | |
| Temozolomide | | |
| Treosulfan | | |
| Triaziquone | | |
| Triethylene Melamine | | |
| ThioTEPA | | Bedford, Abraxis, Teva |
| Triplatin tetranitrate | | |
| Trofosfamide | | |
| Uramustine | | |
| Antimetabolites | | |
| 5-azacytidine | | |
| Flourouracil (5-FU)/Capecitabine | | |
| 6-mercaptopurine | | |
| (Mercaptopurine, 6-MP) | | |
| 6-Thioguanine (6-TG) | Purinethol ® | Teva |
| Cytosine Arabinoside (Cytarabine, | Thioguanine ® | GlaxoSmithKline |
| Ara-C) | | |
| Azathioprine | Azasan ® | AAIPHARMA LLC |
| Capecitabine | XELODA ® | HLR (Roche) |
| Cladribine (2-CdA, 2- | Leustatin ® | Ortho Biotech |
| chlorodeoxyadenosine) | | |
| 5-Trifluoromethyl-2′-deoxyuridine | | |
| Fludarabine phosphate | Fludara ® | Bayer Health Care |
| Floxuridine (5-fluoro-2) | FUDR ® | Hospira, Inc. |
| Methotrexate sodium | Trexall | Barr |
| Pemetrexed | Alimta ® | Lilly |
| Pentostatin | Nipent ® | Hospira, Inc. |
| Raltitrexed | Tomudex ® | AstraZeneca |
| Tegafur | | |
| Aromatose Inhibitor | | |
| Ketoconazole | | |
| Glucocorticoids | | |
| Dexamethasone | Decadron ® Dexasone, | Wyeth, Inc. |
| | Diodex, Hexadrol, Maxidex | |
| Prednisolone | | |
| Prednisone | Deltasone, Orasone, Liquid | |
| | Pred, Sterapred ® | |
| Immunotherapeutics | | |
| Alpha interferon | | |
| Angiogenesis Inhibitor | Avastin ® | Genentech |
| IL-12→ Interleukin 12 | | |
| IL-2→ Interleukin 2 (Aldesleukin) | Proleukin ® | Chiron |
| Receptor Tyrosine Kinasc Inhibitors | | |
| AMG386 | | Amgen |
| Axitinib ((AG-013736) | | Pfizer, Inc |
| Bosutinib (SKI-606) | | Wyeth |
| Brivanib alalinate (BMS-582664) | | BMS |
| Cediranib (AZD2171) | Recentin | AstraVeneca |
| Dasatinib (BMS-354825) | Sprycel ® | Bristol-Myers Squibb |
| Imatinib mesylate | Gleevec | Novartis |
| Lestaurtinib (CEP-701) | | Cephalon |
| Motesanib diphosphate (AMG-706) | | Amgen/Takeda |
| Nilotinib hydrochloride monohydrate | Tasigna ® | Novartis |
| Pazopanib HCL (GW786034) | Armala | GSK |
| Semaxanib (SU5416) | | Pharmacia, |
| Sorafenib tosylate | Nexavar ® | Bayer |
| Sunitinib malate | Sutent ® | Pfizer, Inc. |
| Vandetanib (AZD647) | Zactima | AstraZeneca |
| Vatalanib; PTK-787 | | Novartis; Bayer Schering Pharma |
| XL184, NSC718781 | | Exelixis, GSK |
| Microtubule-Targeting Agents | | |
| Colchicine | | |
| Docetaxel | Taxotere ® | Sanofi-Aventis US |
| Ixabepilone | IXEMPRA ™ | Bristol-Myers Squibb |
| Larotaxel | | Sanofi-aventis |
| Ortataxel | | Spectrum Pharmaceuticals |
| Nanoparticle paclitaxel (ABI-007) | Abraxane ® | Abraxis BioScience, Inc. |
| Paclitaxel | Taxol ® | Bristol-Myers Squibb |
| Tesetaxel | | Genta |
| Vinblastine sulfate | Velban ® | Lilly |
| Vincristine | Oncovin ® | Lilly |
| Vindesine sulphate | Eldisine ® | Lilly |
| Vinflunine | | Pierre Fabre |
| Vinorelbine tartrate | Navelbine ® | Pierre Fabre |
| mTOR Inhibitors | | |
| Deforolimus (AP23573, MK 8669) | | ARIAD Pharmaceuticals, Inc |
| Everolimus (RAD001, RAD001C) | Certican ®, Afinitor | Novartis |
| Sirolimus (Rapamycin) | Rapamune ® | Wyeth Pharama |
| Temsirolimus (CCI-779) | Torisel ® | Wyeth Pharama |
| Protein Synthesis Inhibitor | | |
| L-asparaginase | Elspar ® | Merck & Co. |
| Somatostatin Analogue | | |
| Octreotide acetate | Sandostatin ® | Novartis |
| Topoisomerase Inhibitors | | |
| Actinomycin D | | |
| Camptothecin (CPT) | | |
| Belotecan | | |
| Daunorubicin citrate | Daunoxome ® | Gilead |
| Doxorubicin hydrochloride | Doxil ® | Alza |
| | Vepesid ® | Bristol-Myers Squibb |
| Etoposide | Etopophos | Hospira, Bedford, Teva Parenteral |
| | | Etc. |
| Irinotecan HCL (CPT-11) | Camptosar ® | Pharmacia & Upjohn |
| Mitoxantrone HCL | Novantrone | EMD Serono |
| Rubitecan | | |
| Teniposide (VM-26) | Vumon ® | Bristol-Myers Squibb |
| Topotecan HCL | Hycamtin ® | GlaxoSmithKline |
| Chemotherapeutic Agents | | |
| Adriamycin, 5-Fluorouracil, Cytoxin, | | |
| Bleomycin, Mitomycin C, Daunomycin, | | |
| Carminomycin, Aminopterin, Dactinomycin, | | |
| Mitomycins, Esperamicins Clofarabine, | | |
| Mercaptopurine, Pentostatin, Thioguanine, | | |
| Cytarabine, Decitabine, Floxuridine, | | |
| Gemcitabine (Gemzar), Enocitabine, | | |
| Sapacitabine | | |
| Hormonal Therapies | | |
| Abarelix | Plenaxis ™ | Amgen |
| Abiraterone acetate | CB7630 | BTG plc |
| Afimoxifene | TamoGel | Ascend Therapeutics, Inc. |
| Anastrazole | Arimidex ® | AstraZeneca |
| Aromatase inhibitor | Atamestane plus toremifene | Intarcia Therapeutics, Inc. |
| | Arzoxifene | Eli Lilly & Co. |
| Asentar; DN-101 | | Novartis; Oregon Health & Science |
| | | Univ. |
| Bicalutamide | Casodex ® | AstraZeneca |
| Buserelin | Suprefact ® | Sanofi Aventis |
| Cetrorelix | Cetrotide ® | EMD Serono |
| Exemestane | Aromasin ® | Pfizer |
| Exemestane | Xtane | Natco Pharma, Ltd. |
| Fadrozole (CGS 16949A) | | |
| Flutamide | Eulexin ® | Schering |
| Flutamide | Prostacur | Laboratorios Almirall, S.A. |
| Fulvestrant | Faslodex ® | AstraZeneca |
| Goserelin acetate | Zoladex ® | AstraZeneca |
| Letrozole | Femara ® | Novartis |
| Letrozole (CGS20267) | Femara | Chugai Pharmaceutical Co., Ltd. |
| Letrozole | Estrochek | Jagsonpal Pharmaceuticals, Ltd. |
| Letrozole | Letrozole | Indchemie Health Specialities |
| Leuprolide acetate | Eligard ® | Sanofi Aventis |
| Leuprolide acetate | Leopril | VHB Life Sciences, Inc. |
| Leuprolide acetate | Lupron ®/Lupron Depot | TAP Pharma |
| Leuprolide acetate | Viador | Bayer AG |
| Megestrol acetate | Megace ® | Bristol-Myers Squibb |
| Magestrol acetate | Estradiol Valerate | Jagsonpal Pharmaceuticals, Ltd. |
| | (Delestrogen) | |
| Medroxyprogesterone acetate | Veraplex | Combiphar |
| MT206 | | Medisyn Technologies, Inc. |
| Nafarelin | | |
| Nandrolone decanoate | Zestabolin | Mankind Pharma, Ltd. |
| Nilutamide | Nilandron ® | Aventis Pharmaceuticals |
| Raloxifene HCL | Evista ® | Lilly |
| Tamoxifen | Taxifen | Yung Shin Pharmaceutical |
| Tamoxifen | Tomifen | Alkem Laboratories, Ltd. |
| Tamoxifen citrate | Nolvadex | AstraZeneca |
| Tamoxifen citrate | Soltamox | EUSA Pharma, Inc. |
| Tamoxifen citrate | Tamoxifen citrate | Sopharma JSCo. |
| | SOPHARMA | |
| Toremifene citrate | Fareston ® | GTX, Inc. |
| Triptorelin pamoate | Trelstar ® | Watson Labs |
| Triptorelin pamoate | Trelstar Depot | Paladin Labs, Inc. |
| Protein Kinase B (PKB) Inhibitors | | |
| Akt Inhibitor ASTEX | | Astex Therapeutics |
| Akt Inhibitors NERVIANO | | Nerviano Medical Sciences |
| AKT Kinase Inhibitor TELIK | | Telik, Inc. |
| AKT DECIPHERA | | Deciphera Pharmaceuticals, LLC |
| Perifosine (KRX0401, D-21266) | | Keryx Biopharmaceuticals, Inc., |
| | | AEterna Zentaris, Inc. |
| Perifosine with Docetaxel | | Keryx Biopharmaceuticals, Inc., |
| | | AEterna Zentaris, Inc. |
| Perifosine with Gemcitabine | | AEterna Zentaris, Inc. |
| Perifosine with Paclitaxel | | Keryx Biopharmaceuticals, Inc, |
| | | AEterna Zentaris, Inc. |
| Protein Kinase-B inhibitor DEVELOGEN | | DeveloGen AG |
| PX316 | | Oncothyreon, Inc. |
| RX0183 | | Rexahn Pharmaceuticals, Inc. |
| RX0201 | | Rexahn Pharmaceuticals, Inc. |
| VQD002 | | VioQuest Pharmaceuticals, Inc. |
| XL418 | | Exelixis, Inc. |
| ZEN027 | | AEterna Zentaris, Inc. |
| Phosphatidylinositol 3-Kinase (PI3K) | | |
| Inhibitors | | |
| BEZ235 | | Novartis AG |
| BGT226 | | Novartis AG |
| CAL101 | | Calistoga Pharmaceuticals, Inc. |
| CHR4432 | | Chroma Therapeutics, Ltd. |
| Erk/PI3K Inhibitors ETERNA | | AEterna Zentaris, Inc. |
| GDC0941 | | Genentech Inc./Piramed |
| | | Limited/Roche Holdings, Ltd. |
| Enzastaurin HCL (LY317615) | Enzastaurin | Eli Lilly |
| LY294002/Wortmannin | | |
| PI3K Inhibitors SEMAFORE | | Semafore Pharmaceuticals |
| PX866 | | Oncothyreon, Inc. |
| SF1126 | | Semafore Pharmaceuticals |
| VMD-8000 | | VM Discovery, Inc. |
| XL 147 | | Exelixis, Inc. |
| XL147 with XL647 | | Exelixis, Inc. |
| XL765 | | Exelixis, Inc. |
| PI-103 | | Roche/Piramed |
| Cyclin-dependent kinase inhibitors | | |
| CYC200, r-roscovitine | Seliciclib | Cyclacel Pharma |
| NSC-649890, L86-8275, HMR-1275 | Alvocidib | NCI |
| TLr9, CD289 | | |
| IMOxine | | Merck KGaA |
| HYB2055 | | Idera |
| IMO-2055 | | Isis Pharma |
| 1018 ISS | | Dynavax Technologies/UCSF |
| PF-3512676 | | Pfizer |
| Enzyme Inhibitor | | |
| Lonafarnib (SCH66336) | Sarasar | SuperGen, U Arizona |
| Anti-TRAIL | | |
| AMG-655 | | Aeteraa Zentaris, Keryx Biopharma |
| Apo2L/TRAIL, AMG951 | | Genentech, Amgen |
| Apomab (fully humanized mAb | | Genentech |
| Other | | |
| Imprime PGG | | Biothera |
| CHR-2797 | AminopeptidaseM1 | Chroma Therapeutics |
| E7820, NSC 719239 | Integrin-alpha2 | Eisai |
| INCB007839 | ADAM 17, TACE | Incyte |
| CNF2024, BIIB021 | Hsp90 | Biogen IdeC |
| MP470, HPK-56 | Kit/Met/Ret | Shering-Plough |
| SNDX-275/MS-275 | HDAC | Syndax |
| Zarnestra, Tipifarnib, R115777 | Ras | Janssen Pharma |
| Volociximab; Eos 200-4, M200 | alpha581 integrin | Biogen IdeC; Eli Lilly/UCSF/PDL |
| | | BioPharma |
| Apricoxib (TP2001) | COX-2 Inhibitor | Daiichi Sankyo; Tragara Pharma |

1. A method of treating a subject with a malignant tumor, the method comprising co-administering to the subject:
either
i) an effective amount of an anti-estrogen agent, or
ii) an effective amount of a receptor tyrosine kinase inhibitor; and
either
iii) an effective amount of a bispecific anti-ErbB2/anti-ErbB3 antibody, or
iv) an effective amount of a bispecific anti-ErbB2/anti-ErbB3 antibody and an effective amount of trastuzumab.

2.-5. (canceled)

6. The method of claim 1, wherein all effective amounts are either mouse effective amounts or human effective amounts.

7.-8. (canceled)

9. The method of claim 1, wherein the co-administration to the subject does not create a drug-drug interaction-mediated toxicity in the subject.

10. The method of claim 1, wherein the co-administration to the subject creates a substantially additive or superadditive effect.

11. The method of claim 1, wherein the anti-estrogen agent is an estrogen receptor antagonist or an aromatase inhibitor.

12. The method of claim 11, wherein the estrogen receptor antagonist is fulvestrant or tamoxifen.

13. The method of claim 11, wherein the aromatase inhibitor is letrozole, exemestane, anastrozole, aminoglutethimide, testolactone, vorozole, formestane, or fadrozole.

14. The method of claim 13, wherein the aromatase inhibitor is letrozole.

15. The method of claim 1, wherein the bispecific anti-ErbB2/anti-ErbB3 antibody comprises the amino acid sequence set forth in SEQ ID NO:1.

16. The method of claim 1, wherein the bispecific anti-ErbB2/anti-ErbB3 antibody is chosen from the group consisting of A5-HSA-ML3.9, ML3.9-HSA-A5, A5-HSA-B1D2, B1D2-HSA-A5, B12-HSA-B1D2, B1D2-HSA-B12, A5-HSA-F5B6H2, F5B6H2-HSA-A5, H3-HSA-F5B6H2, F5B6H2-HSA-H3, F4-HSA-F5B6H2, F5B6H2-HSA-F4, B1D2-HSA-H3, and H3-HSA-B1D2.

17. The method of claim 1, wherein the receptor tyrosine kinase inhibitor is erlotinib, afatinib, dasatinib, gefitinib, imatinib, pazopinib, lapatinib, sunitinib, nilotinib or sorafenib.

18. The method of claim 1, wherein the receptor tyrosine kinase inhibitor is lapatinib.

19. The method of claim 18 wherein the lapatinib is administered via a dosing regimen comprising a 14-day dosing schedule, and wherein the lapatinib is administered intermittently.

20. The method of claim 19, wherein the lapatinib is administered on days 1 to 3, 1 to 4, 1 to 5, 1 to 6, or 1 to 7 of the 14-day dosing schedule.

21. The method of claim 20, wherein the lapatinib is administered on days 1 to 5 of the 14-day dosing schedule.

22. The method of claim 19, wherein the lapatinib is administered at a dose that is between 2000 and 9000 mg/d.

23. The method of claim 22, wherein the dose is 3000 mg/day.

24. The method of claim 19, wherein the dose of lapatinib administered on day 1 of the 14-day dosing cycle comprises a loading dose.

25. The method of claim 1, further comprising co-administering an effective amount of capecitabine and/or cisplatin.

26.-41. (canceled)

42. An aqueous solution comprising a bispecific anti-ErbB2/anti-ErbB3 antibody at a first concentration and either i) an anti-estrogen agent at a second concentration or ii) a receptor tyrosine kinase inhibitor at a third concentration, wherein each concentration is an effective concentration and when the aqueous solution is blood plasma in a subject, the subject does not experience a toxicity that is sufficiently harmful to require a change in a therapy being administered to the subject, which toxicity is mediated by a drug-drug interaction in the subject between the bispecific anti-ErbB2/anti-ErbB3 antibody and the anti-estrogen agent or the receptor tyrosine kinase inhibitor.

43. The aqueous solution of claim 42, wherein the anti-estrogen agent is fulvestrant or tamoxifen.

44. The aqueous solution of claim 42, wherein the bispecific anti-ErbB2/anti-ErbB3 antibody comprises the amino acid sequence set forth in SEQ ID NO:1.

45. The aqueous solution of claim 42, wherein the bispecific anti ErbB3, anti-ErbB2 antibody is chosen from the group consisting of A5-HSA-ML3.9, ML3.9-HSA-A5, A5-HSA-B1D2, B1D2-HSA-A5, B12-HSA-B1D2, B1D2-HSA-B12, A5-HSA-F5B6H2, F5B6H2-HSA-A5, H3-HSA-F5B6H2, F5B6H2-HSA-H3, F4-HSA-F5B6H2, F5B6H2-HSA-F4, B1D2-HSA-H3, and H3-HSA-B1D2.

46. The aqueous solution of claim 42, wherein the receptor tyrosine kinase inhibitor is chosen from the group consisting essentially of erlotinib, afatinib, dasatinib, gefitinib, imatinib, pazopinib, lapatinib, sunitinib, nilotinib and sorafenib.

47. The aqueous solution of claim 42, wherein the receptor tyrosine kinase inhibitor is lapatinib.

48. A method of inhibiting the growth of a malignant tumor comprising tumor cells, said method comprising contacting the tumor cells with the aqueous solution of claim 42.

49. The method of claim 1, wherein the estrogen receptor antagonist is fulvestrant or tamoxifen.

50.-63. (canceled)

64. A method of treating a subject with a malignant tumor, the method comprising co-administering to the subject, 1) an effective amount of a bispecific anti-ErbB2/anti-ErbB3 antibody, 2) an effective amount of trastuzumab, 3) an effective amount of cisplatin, and 4) an effective amount of capecitabine.

65. A method of treating a subject with a malignant tumor, the method comprising co-administering to the subject, 1) an effective amount of a bispecific anti-ErbB2/anti-ErbB3 antibody, 2) an effective amount of trastuzumab, and 3) an effective amount of nab-paclitaxel.


SEQUENCE LISTING (1 sequence)

SEQ ID NO: 1 - protein, 1095 aa, Artificial Sequence
  Synthetic Construct
QVQLQESGGGLVKPGGSLRLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSSASTGGGGSGGGGSGGGGSQSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTVLGAASDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFQAETFTFHADICTLSEKERQIKKQTALVELVKHKPKATKEQLKAVMDDFAAFVEKCCKADDKETCFAEEGKKLVAASQAALGLAAALQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEWLGVWGQGTLVTVSSGGGGSSGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLG